(19) United States
(12) Patent Application Publication
(10) Pub. No.: US 2026/0048067 A1
(43) Pub. Date: Feb. 19, 2026

(54) N-(1-((R)-1-Acryloylazepan-3-YL)-7-Chloro-6-(((R)-Tetrahydrofuran-3-YL)OXY)-1H-BENZO[D]Imidazol-2-YL)-2-Methylisonicotinamide (NX-019) for Use in the Treatment of EGFR Mutant Cancer

(71) Applicant: Nalo Therapeutics, Inc., Palo Alto, CA (US)

(72) Inventors: Guy Gammon, Del Mar, CA (US); Keith Wilson, Palo Alto, CA (US)

(21) Appl. No.: 19/104,135

(22) PCT Filed: Aug. 15, 2023
(86) PCT No.: PCT/US2023/030297

§ 371 Date: Feb. 14, 2025

Related U.S. Application Data
(60) Provisional application No. 63/398,192, filed on Aug. 15, 2022.
(60) Provisional application No. 63/385,388, filed on Nov. 29, 2022.
(60) Provisional application No. 63/484,791, filed on Feb. 14, 2023.
(60) Provisional application No. 63/504,416, filed on May 25, 2023.

(51) Int. Cl. A61K 31/55; A61K 9/00; A61P 35/00

(52) U.S. Cl. CPC A61K 31/55; A61K 9/0053; A61P 35/00

(57) ABSTRACT

NX-019 is a potent, orally administrable small molecule inhibitor of EGFR that shows selective inhibition activity for EGFR having one or more mutations. This present disclosure provides methods of treating EGFR mutant cancer which include orally administering a therapeutically effective amount that is a daily dose of 37.5 mg to 450 mg of NX-019 to a human patient having cancer with at least one mutation in the epidermal growth factor receptor (EGFR) gene. Also provided are methods of treating cancer in a patient having CNS metastasis which includes orally administering a therapeutically effective amount that is a daily dose of 37.5 mg to 450 mg of NX-019 to a human patient having cancer with CNS metastasis and at least one mutation in the epidermal growth factor receptor (EGFR) gene. In some embodiments, the EGFR mutant cancer is NSCLC.

### 1. BACKGROUND

Epidermal growth factor receptor (EGFR) mutations occur in a wide range of tumor types including head and neck cancer and colon cancer; however, the great majority of cancers with EGFR mutations are in patients with non-small cell lung cancer (NSCLC). Lung cancer is the leading cause of cancer mortality worldwide, with NSCLC comprising the vast majority (85%) of all lung malignancies. While the prognosis for NSCLC is considerably better than for small cell lung cancer (SCLC), the overall 5-year survival rate is still very low (approximately 15%) due to the advanced stage at which most patients are diagnosed and the inherent molecular heterogeneity of the disease. EGFR mutations in non-small cell lung cancer (NSCLC) are a heterogeneous group of mutations including common mutations, L858R and exon 19 deletions, and rare mutations exon 20 insertion (ex20ins), G719X, S768I, and L861Q mutations (Riess JW, et al. J Thorac Oncol, 2018; 13(10): 1560-1568).

CNS involvement, brain metastases and/or leptomeningeal disease, is common in patients with EGFR mutant NSCLC and occurs in 25-50% of patient's journeys and has been associated with worse clinical outcomes. Despite the activity and increased CNS penetration of later generation EGFR inhibitors, management of CNS disease remains a clinical challenge. Most approved EGFR inhibitors fail to address the unmet needs in patients with CNS disease due to limited CNS activity (ErnaniV, et al. J Oncol Pract, 2019; 15(11): 563-570 and D′Aiello A, et al. Cancers, 2023; 15(3): 84).

The development of targeted therapies for lung cancer such as NSCLC has primarily focused on tumors displaying genetic alterations including specific oncogenic drivers, namely mutations in epidermal growth factor receptor (EGFR), rearrangements of anaplastic lymphoma kinase (ALK), KRAS mutations, BRAF V600E substitution, MET exon 14 mutations, and fusion genes involving ROS proto-oncogene 1, rearranged during transfection (RET), and neurotrophic tyrosine receptor kinase. Three generations of tyrosine kinase inhibitors (TKIs) have been developed for cancers with the most frequently observed EGFR mutations. Other oncogenic drivers in the EGFR family of receptor tyrosine kinases include insertions in the exon 20 gene of EGFR, for which approved anti-cancer therapeutics are needed.

Brain and bone metastases are two of the most common sites for NSCLC patients. Bone metastasis is associated with a poor progression-free survival (PFS) time, and brain metastasis is associated with a poor overall survival (OS) time with the number of metastatic organ sites associated with a poorer PFS and OS time and poor quality of life. Metastases to all sites (including bone and brain) are increased in EGFR mutations (EGFRm)+ tumors because these patients tend to live longer due to effective therapies such as EGFR kinase inhibitors.

Many cancer patients with EGFR mutations derive no clinical benefit from current therapies. Thus, the development of new therapies for the treatment of cancers associated with EGFR mutations are of great interest.

### 2. SUMMARY

This disclosure provides methods of treating a patient for EGFR mutant cancer using an EGFR inhibitor compound. NX-019 is a potent, orally administrable small molecule inhibitor of EGFR that shows selective inhibition activity for EGFR having one or more mutations.

This disclosure describes a clinical study of patients having EGFR mutant cancer to demonstrate the efficacy and safety of NX-019 when it is administered to the patients according to the described clinical protocol. This is a 2-part, first-in-human, international, open-label study designed to evaluate single-agent NX-019 in patients with EGFR-mutant, locally advanced or metastatic NSCLC that has progressed following prior treatment. Patients with ECOG performance status 0-2 and EGFR mutant cancers including stable, asymptomatic brain metastases are eligible for Dose Escalation. Patients with known EGFR C797S mutations and secondary drivers, e.g., MET amplification and RET fusions, are excluded. Primary endpoints are to determine recommended phase 2 dose (RP2D), maximum tolerated dose (MTD), PK, safety and tolerability (part 1), and to evaluate preliminary evidence of activity, antitumor activity at the RP2D by RECIST v1.1 and RANO-BM, and confirm safety profile (part 2). Secondary endpoints are to evaluate pharmacokinetics (PK) and antitumor activity by RECIST v1.1 (part 1) and PK, safety, tolerability, and CNS antitumor activity (part 2). Dose escalation will utilize a 3+3 design with up to 6 patients per cohort in part 1. Part 2 will enroll patients in 5cohorts (n=10 or 29 each), including patients with NSCLC who have (1) new or progressing CNS metastases including leptomeningeal disease after treatment with osimertinib, (2) active CNS disease and ex20ins mutations. (3) ex20ins mutations without CNS disease and (4) other rare mutations, based on a Simon 2-stage design. First patient was treated in October 2022 and enrollment is ongoing.

Accordingly, the present disclosure provides methods of treating EGFR mutant cancer which include orally administering a therapeutically effective amount that is a daily dose of 37.5 mg to 450 mg of NX-019 to a human patient having cancer with at least one mutation in the epidermal growth factor receptor (EGFR) gene.

In some embodiments, the patient has systemic EGFR mutant cancer. In some embodiments, the patient has NSCLC. In some embodiments, the patient's cancer has central nervous system (CNS) lesion(s).

Accordingly, the present disclosure provides methods of treating cancer in a patient having CNS metastasis which include orally administering a therapeutically effective amount that is a daily dose of 37.5 mg to 450 mg of NX-019 to a human patient having cancer with CNS metastasis and at least one mutation in the epidermal growth factor receptor (EGFR) gene.

In some embodiments, the patient's cancer does not have an insertion in Exon 20 of the EGFR gene. In some embodiments, the patient's cancer has an insertion in Exon 20 of the EGFR gene. In some embodiments, the EGFR mutant cancer is NSCLC.

### 3. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1, panels A-C, illustrate the activity of NX-019 in a mouse model of brain cancer (PC9-GFP) harboring an EGFR exon 19 insertional mutation (DelE746-A750) in the brain of the mice. NX-019 caused complete regression of tumors as shown by the graph of tumor reduction (panel A), the photos of the GFP fluorescent tumors at Day 22 in control versus treated mice (75mg/kg) (panel B), and the down-regulation of phosphorylated EGFR in the tumors at time points post dose (175 mg/kg) (panel C).

FIG. 2, panel A illustrates the GFP fluorescent tumor at Day 22 in treated mice at a higher dosage (125 mg/kg) versus the control in FIG. 1. Panel B shows body weight of treated mice is consistent throughout the duration of treatment.

FIG. 3 depicts potency of NX-019 toward EGFR mutations in Ba/F3 of cancer cell lines expressing wildtype or mutant EGFR.

FIG. 4, panels A-C illustrate selective inhibition of mutant EGFR over wildtype EGFR. Panel A shows Western blot analysis of normal skin and exon 20 ins (D770_N771insSVD) tumors. Panel B shows expression levels of phosphorylated EGFR proteins decreases in response to increased dosage of NX-019. β-actin protein level is used as an internal control. Panel A shows phosphorylated EGFR levels as quantitated and normalized to total EGFR and β-actin protein levels. Panel C shows selectivity of NX-019 toward EGFR Exon 20 ins mutations.

FIG. 5, panels A-B illustrate tumor regression in EGFR exon 20 ins xenografts. Panel A shows efficacy of NX-019 in PDX model of Exon 20 ins (LU0387-H773_V774insNPH). Panel B shows body weight of treated mice has little or no change throughout the duration of treatment.

FIG. 6, panels A-C, illustrate NX-019 shows significant CNS/brain exposure in animal models. Panel A shows NX-019 brain and plasma exposure ratios in mouse and rat. Shown arc the area under curve (AUC) of brain/plasma ratio in rat, AUC brain/plasma ratio in mouse, Cₘₐₓbrain/plasma ratio in rat, and Cₘₐₓbrain/plasma ratio in mouse. Vertical dotted line indicates a ratio of 1:1 or higher. Panel B shows NX-019 Kp,uu values in mouse and rat. Kp,uu is the ratio of unbound drug in brain versus plasma. Panel C shows calculated plasma free fraction/CSF ratio in cynomolgus monkey, dog, and rat. Panel D shows NX-019 Kp,uu values in rat, dog, and monkey. Panel E shows Kpuu_{CSF} values for NX-019 compared to Osimertinib.

FIG. 7 is a flowchart illustrating a human study of NX-019 in patients with advanced, EGFR mutant cancer.

FIG. 8 shows NX-019 has activity in a broad range of mutations.

FIG. 9 is another flowchart illustrating a human study of NX-019 in patients with advanced, EGFR mutant cancer

### 4. DETAILED DESCRIPTION

### 4.1. Methods of Treating EGFR Mutant Cancer

The present disclosure provides a method of treating EGFR mutant cancer in a patient is need thereof. The methods of this disclosure described herein can be extended to a variety of cancers, particularly NSCLC. Examples of cancers related to the methods described herein include, but are not limited to, breast cancer, lung cancer, head and neck cancer, and colon cancer.

In some embodiments, the patient has lung cancer. In some embodiments, the lung cancer is non-small cell lung cancer (NSCLC).

The term “cancer” is meant to refer to a malignant neoplasm, which is characterized by uncontrolled cell proliferation where cells have lost their normal regulatory controls that would otherwise govern the rate of cell growth. These unregulated, dividing cells can spread throughout the body and invade normal tissues in a process referred to as “metastasis.”

In some embodiments, the cancer is human epidermal growth factor receptor 2 positive (HER2⁺).

### 4.1.1. EGFR Mutant Cancer

In some embodiments, the patient has a cancer having at least one mutation in the epidermal growth factor receptor (EGFR) gene, also referred to as an EGFR mutation, i.e., “(EGFRm)+”. In such cases, the cancer may be referred to as an EGFR mutant (EGFRm) cancer.

These EGFR mutations are oncogenic, promoting cell proliferation, survival and anti-apoptotic signals, angiogenesis, and potentially, metastases. The signaling networks in EGFR-mutated cells depend on a functionally mutated EGFR for their survival, rendering them “addicted” to the activated receptor.

The landscape of EGFR mutations, e.g., in NSCLC, includes the classical EGFRm such as L858R and exon 19 deletions, which stabilize an active dimer confirmation of the receptor leading to receptor activation and cellular oncogenic transformation. The classical mutations account for approximately 88% of EGFRm. The other set of EGFRm, comprising ˜12% of EGFRm in NSCLC, arc the rare mutations, which are a heterogeneous set of mutations including mutations in exons 18, 19, 20, and 21 of EGFR gene. These mutations, like the classical mutations, are in or near the EGFR activation site or activation domain region of the receptor and lead to an activated, non-ligand dependent EGFR. These activating mutations in NSCLC are known, in many cases, to produce worse patient outcomes.

In some embodiments, the EGFRm cancer includes an insertion mutation in the EGFR gene. In some embodiments, the insertion mutation is in Exon 20 of the EGFR gene. In other embodiments, the insertion mutation is not an insertion in Exon 20 of the EGFR gene. In some embodiments, the insertion mutation is in Exon 19 of the EGFR gene.

In some embodiments, the EGFRm cancer includes a deletion in the EGFR gene. In some embodiments, the deletion is in Exon 19 of the EGFR gene.

In some embodiments, the EGFRm cancer includes a point mutation in the EGFR gene. In some embodiments, the point mutation is located at one or more of amino acid positions L858,G719, L861, S768, and E709 of the EGFR gene. In some embodiments, the point mutation is located at G719 of the EGFR gene. In some embodiments. the point mutation is located at L861of the EGFR gene. In some embodiments, the point mutation is located at S768 of the EGFR gene. In some embodiments, the point mutation is located at E709 of the EGFR gene.

In some embodiments, the EGFRm cancer includes a frameshift mutation in the EGFR gene.

In some embodiments of the methods of this disclosure, the patient has previously been treated with an EGFR-targeted therapy. In some embodiments, the patient has previously been treated with osimertinib.

In some embodiments of the methods of this disclosure, the patient has not previously been treated with an EGFR-targeted therapy targeting an insertion in Exon 20.

4.1.1. Patients having Systemic Tumors

In some embodiments, the cancer being treated is locally advanced.

In some embodiments, the cancer being treated is metastatic. In some embodiments, the patient's cancer has bone metastasis.

The methods of this disclosure include administration of a therapeutically effective amount of NX-019, where the patient exhibits regression of systemic tumors.

In some embodiments, the patient's cancer is NSCLC having at least one EGFRm (e.g., as described herein).

4.1.2. Patients having CNS Lesions

Many patients develop a central nervous system (CNS) lesion over the course of their disease. A central nervous system (CNS) lesion can be identified as an abnormality on an imaging test, such as magnetic resonance imaging (MRI) or computerized tomography (CT).

In some embodiments, the CNS lesion includes at least one EGFRm (e.g., as described herein).

In some embodiments, the patient has a CNS lesion that is a metastatic brain tumor.

In some embodiments, the patient has a CNS lesion that is a NSCLC brain metastasis.

The methods of this disclosure include administration of a therapeutically effective amount of NX-019 to provide sufficient brain penetration of the compound and regression of NSCLC brain metastases in the patient.

In some embodiments, the patient's cancer has NSCLC brain metastases having at least one EGFRm (e.g., as described herein).

### 4.2. EGFR Inhibitor Compound and Compositions

An EGFR inhibitor compound as described herein, and compositions including such a compound which function to inhibit activity of an EGFR, such as an EGFR mutant, are useful in the methods of this disclosure.

In some embodiments, the EGFR inhibitor compound is of the formula:

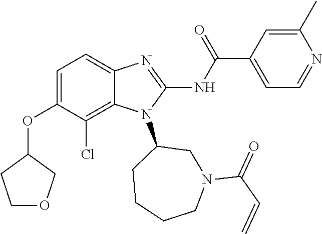 [SMILES: C=CC(=O)N1CCCC[C@@H](n2c(NC(=O)c3ccnc(C)c3)nc3ccc(OC4CCOC4)c(Cl)c32)C1]

or a single enantiomer, a racemic mixture, a mixture of diastereomers, or an isotopic variant thereof, or a pharmaceutically acceptable salt, or solvate thereof.

The phrase “a single enantiomer, a racemic mixture, a mixture of diastereomers, or an isotopic variant thereof; or a pharmaceutically acceptable salt, or solvate thereof” has the same meaning as the phrase “(i) a single enantiomer, a racemic mixture, a mixture of diastereomers, or an isotopic variant of the compound referenced therein; (ii) a pharmaceutically acceptable salt, or solvate of the compound referenced therein; or (iii) a pharmaceutically acceptable salt, or solvate of a single enantiomer, a racemic mixture, a mixture of diastereomers, or an isotopic variant of the compound referenced therein.

An EGFR inhibitor compound containing a nitrogen-containing heteroaryl group may be basic in nature and may react with a variety of inorganic and organic acids to form the corresponding pharmaceutically acceptable salts. The acids that can be used to prepare pharmaceutically acceptable acid addition salts of such basic compounds are those that form non-toxic acid addition salts, i.e., salts containing pharmacologically acceptable anions. Inorganic acids commonly employed to form such salts include hydrochloric, and related inorganic acids. Organic acids commonly employed to form such salts include acetic acid, formic acid, and related organic acids. Such pharmaceutically acceptable salts thus include chloride, acetate, formate, and related salts.

In some embodiments, the EGFR inhibitor compound is NX-019, a compound having the structure:

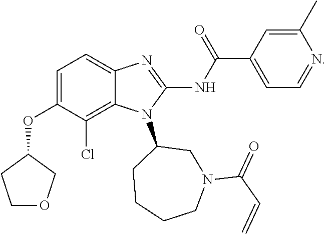 [SMILES: C=CC(=O)N1CCCC[C@@H](n2c(NC(=O)c3ccnc(C)c3)nc3ccc(O[C@H]4CCOC4)c(Cl)c32)C1]

The synthesis and EGFR inhibitor activity of NX-019 and related compounds is described in, e.g., U.S. Pat. No. 10,005,765, the disclosure of which is hereby incorporated by reference.

In some embodiments of the methods of this disclosure, the compound NX-019 is

provided and administered as a free base form. In some embodiments of the methods of this disclosure, the compound NX-019 is provided and administered in the form of a pharmaceutically acceptable salt.

An EGFR inhibitor compound described herein can be present in various forms including crystalline, powder and amorphous forms of the compound, pharmaceutically acceptable salts, including, for example, polymorph, pseudopolymorph, solvate, hydrate, unsolvated polymorph (including anhydrates), conformational polymorph, and amorphous forms of the compound, as well as mixtures thereof.

The compounds described herein may exist as solvates, especially hydrates, and unless otherwise specified, all such solvates and hydrates are intended. Hydrates may form during manufacture of the compounds or compositions comprising the compounds, or hydrates may form over time due to the hygroscopic nature of the compounds. Compounds of the present technology may exist as organic solvates as well, including alcohol solvates. Suitable solvents include, but are not limited to, water, methanol, ethanol, n-propanol, isopropanol, and acetic acid. The identification and preparation of any particular solvate is within the skill of the ordinary artisan of synthetic organic or medicinal chemistry.

In some embodiments, the compounds described herein are present in a solvate form. In some embodiments, the compounds described herein are present in a hydrate form when the solvent component of the solvate is water.

In some embodiments of the method, the EGFR inhibitor compound is an isotopically labeled compound (also referred to as an isotopic variant of the compound, or an isotopologue) which is identical to the compound described herein, except that one or more atoms are replaced by an atom having an atomic mass or mass number different from the atomic mass or mass number usually found in nature. The compounds of the present disclosure may also contain unnatural proportions of atomic isotopes at one or more atoms that constituted such compounds. Examples of isotopes that can be incorporated into compounds described herein include isotopes of hydrogen, carbon, nitrogen, oxygen, and chlorine, such as ²H(“D”), ³H, ¹³C, ¹⁴C, ¹⁵N, ¹⁸O, ¹⁷O, and ³⁶Cl, respectively. For example, a compound described herein can have one or more H atoms replaced with deuterium. Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure. Isotopically-labeled compounds can generally be prepared by following analogous procedures to those disclosed, by substituting an isotopically-labeled reagent for a non-isotopically-labeled reagent.

### 4.2.1. Pharmaceutical Compositions

In some embodiments of the methods of treating cancer, the EGFR inhibitor compound of the present disclosure that is administered to the subject may be composed in a pharmaceutical composition. The pharmaceutical composition can include the compound as described herein, or a pharmaceutically acceptable salt thereof, in a therapeutically effective amount, and at least one pharmaceutically acceptable excipient.

The term “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (i.e., the compound(s) in the pharmaceutical composition is pharmaceutical grade). Pharmaceutical compositions can be designed for administration to subjects or patients in need thereof via an oral route of administration.

The terms “pharmaceutically acceptable excipient,” “pharmaceutically acceptable diluent.” “pharmaceutically acceptable carrier.” and “pharmaceutically acceptable adjuvant” are used interchangeably and refer to any ingredient other than the EGFR inhibitor compound described herein (for example, a vehicle capable of suspending or dissolving the active compound, or any other convenient pharmaceutically acceptable carriers, excipients, diluent, adjuvant or additives) and having the properties of being substantially nontoxic and non-inflammatory in a patient. The phrase “pharmaceutically acceptable excipient” includes both one and more than one such excipient, diluent, carrier, and/or adjuvant. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, dispensing, or dispersing agents, sweeteners, and waters of hydration.

The pharmaceutical composition may be formulated according to any convenient methods, and may be prepared in various forms for oral administration such as tablets, pills, powders, capsules, syrups, emulsions and microemulsions.

### 4.3. Administration Methods

An aspect the methods of treating a cancer of this disclosure is orally delivering the EGFR inhibitor compound or composition to the patient. A compound can be systemically administered, e.g., orally, in a pharmaceutical composition in combination with a pharmaceutically acceptable vehicle such as an inert diluent or an assimilable carrier. The term “patient” refers to a human subject.

The phrase “therapeutically effective amount” refers to the amount of a compound that, when administered to a patient for treating a disease, condition, or disorder, is sufficient to effect treatment of the disease, condition, or disorder. The “therapeutically effective amount” may vary depending on the disease and its severity and the age, weight, etc., of the subject to be treated.

The terms “treating” and “treatment” refer to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or conditions. Accordingly, the terms do not require cure or complete remission of disease and encompass obtaining any clinically desired pharmacologic and/or physiologic effects.

It will be appreciated by the skilled worker that methods disclosed herein are disclosed also as their corresponding “Swiss-type” or “EPC2000” equivalent. Thus a method of treating cancer in a patient, wherein the patient's cancer has at least one mutation in the epidermal growth factor receptor (EGR) gene, the method comprising orally administering to the patient daily doses of NX-019 or a pharmaceutically acceptable salt thereof, or a method of treating cancer in a patient having CNS metastasis, the method comprising orally administering to the patient daily doses of NX-019 or a pharmaceutically acceptable salt thereof, would be understood also to disclose the use of NX-019 or a pharmaceutically acceptable salt thereof in the manufacture of a medicament for treating cancer in a patient, wherein the patient's cancer has at least one mutation in the epidermal growth factor receptor (EGR) gene or the use of NX-019or a pharmaceutically acceptable salt thereof in the manufacture of a medicament for treating cancer in a patient having CNS metastasis, respectively. Likewise, disclosure of the above methods would be understood to disclose NX-019 or a pharmaceutically acceptable salt thereof for use in a method of treating cancer in a patient, wherein the patient's cancer has at least one mutation in the epidermal growth factor receptor (EGR) gene. the method comprising orally administering to the patient daily doses of NX-019 or a pharmaceutically acceptable salt thereof, or a method of treating cancer in a patient having CNS metastasis, the method comprising orally administering to the patient daily doses of NX-019 or a pharmaceutically acceptable salt thereof.

### 4.3.1. Dosage

In some embodiments of the method, the effective amount of the compound is achieved by oral administration of a dosage regimen including a daily dose of 37.5 to 450 mg of NX-019 or a pharmaceutically acceptable salt thereof.

In some embodiments, the dose is administered as a single daily dose (QD).

In some embodiments, the daily oral dose of NX-019 or a pharmaceutically acceptable salt thereof, is at least 75 mg. In some embodiments, the daily oral dose of NX-019 or a pharmaceutically acceptable salt thereof. is at least 150 mg. In some embodiments, the daily oral dose of NX-019 or a pharmaceutically acceptable salt thereof. is at least 300 mg. In some embodiments, the daily oral dose of NX-019 or a pharmaceutically acceptable salt thereof, is at least 375 mg.

In some embodiments, the daily oral dose of NX-019 or a pharmaceutically acceptable salt thereof, is 75-150 mg.

In some embodiments, the daily oral dose of NX-019 or a pharmaceutically acceptable salt thereof, is 150-300 mg.

In some embodiments, the daily oral dose of NX-019 or a pharmaceutically acceptable salt thereof, is 37.5 mg. In some embodiments, the daily oral dose of NX-019 or a pharmaceutically acceptable salt thereof, is 75 mg. In some embodiments, the daily oral dose of NX-019 or a pharmaceutically acceptable salt thereof, is 150 mg. In some embodiments, the daily oral dose of NX-019 or a pharmaceutically acceptable salt thereof, is 300 mg. In some embodiments, the daily oral dose of NX-019 or a pharmaceutically acceptable salt thereof, is 375 mg. In some embodiments, the daily oral dose of NX-019 or a pharmaceutically acceptable salt thereof, is 450 mg.

In some embodiments, the NX-019 is administered in a free base form composed in a pharmaceutical composition.

In some embodiments, the NX-019 or a pharamceutically acceptable salt thereof, is administered composed in a pharmaceutical composition.

In some embodiments of the method, the daily dose is administered orally to the patient for as long as needed. In some embodiments, the daily dose is administered for one week or more, such as two weeks or more, three weeks or more, or four weeks or more. In some embodiments, the daily dose is administered for 28 days. In some embodiments, the course of treatment can be repeated as needed. In some embodiments, the daily dose is administered for up to 3 months, up to 6 months, up to one year, up to two years, up to three years, up to 4 years, or up to 5 years.

The compounds of the present disclosure can also be co-administered with one or more additional therapeutic compounds. “Co-administration” of compounds includes one or more compounds administered substantially simultaneously with the EGFR inhibitor compound of this disclosure, whether or not in the same pharmaceutical composition, or sequentially. In some embodiments of the method of this disclosure, the EGFR inhibitor compound disclosed herein is administered or combined with other anticancer therapies, such as internal or external radiation. surgery, and chemotherapies.

### 4.3.2. Patient Selection/Condition

An aspect of this disclosure is treating a patient for an EGFR-mediated cancer. The present disclosure provides method of treating cancer having at least one mutation in the epidermal growth factor receptor (EGFR) gene.

In some embodiments, the patient has an EGFR-mediated cancer selected from lung cancer, head and neck cancer, and colon cancer. In some embodiments, the patient has lung cancer. In some embodiments, the lung cancer is NSCLC.

In some embodiments, the patient's cancer is locally advanced. In some embodiments, the patient's cancer is metastatic. In some embodiments, the patient has systemic NSCLC.

The experimental section of this disclosure describes several inclusion and exclusion criteria for a clinical study which can be applied in selecting a patient to receive treatment according to the methods of this disclosure. In some embodiments, the inclusion criteria includes patients with histologically confirmed, locally advanced, or metastatic EGFR-mutant cancer that has progressed on or is intolerant to all standard therapy; patients with non-small cell lung cancer (NSCLC) harboring a mutation that is sensitive to osimertinib must have received osimertinib; patients with a measurable disease; baseline MRI of the brain required; patients with adequate organ and bone marrow function; patients with an Eastern Cooperative Oncology Group (ECOG) performance status of ≤2;and patients ≥18 years of age. In some embodiments the exclusion criteria applies to patients with any of: a known C797X EGFR mutations or known second driver of disease; a severe or unstable medical condition including cardiac conditions, uncontrolled diabetes or unstable psychiatric condition; a history of interstitial lung disease, radiation pneumonitis which required systemic steroid therapy. or other significant lung disease; known infection requiring systemic therapy, including HIV, HBV, HCV; active gastrointestinal disease that may impact drug absorption; using a strong CYP3A inhibitor or inducer; or any condition, including significant skin or nail disease, that in the Investigator's opinion would place the patient at an unacceptable risk or not allow the patient to comply with the protocol.

In some embodiments, the patient's cancer has asymptomatic leptomeningeal disease. In some embodiments, the patient's cancer is human epidermal growth factor receptor 2 positive (HER2⁺). In some embodiments, the patient has previously been treated with an EGFR-targeted therapy. In some embodiments, the patient has previously been treated with osimertinib.

In some embodiments, the patient exhibits an improvement in one or more primary or secondary outcome measures of efficacy (e.g., as described herein) after receiving treatment according to the methods of this disclosure. In some embodiments, the patient exhibits systemic tumor regression after receiving treatment.

In some embodiments, treatment according to the methods of this disclosure increases the overall survival (OS) time of a patient population as compared to a control patient population without the administration.

In some embodiments, treatment according to the methods of this disclosure increases the progression-free survival (PFS) time of a patient population as compared to a control patient population without the administration.

4.3.2.1 Patient having CNS metastasis

Aspects of this disclosure include treating a patient's cancer that has central nervous system (CNS) metastasis and at least one mutation in the epidermal growth factor receptor (EGFR) gene.

In some embodiments, the patient is identified as having such as EGFRm cancer with CNS disease, e.g., having CNS lesion(s) such as brain metastases. In some embodiments, the patient has measurable CNS lesion(s) with ≥5 mm diameter as determined by MRI (e.g., as described herein). In some embodiments, the cancer is NSCLC with CNS disease.

In some embodiments, the patient's cancer with CNS disease is identified as having EGFR ex20ins-mutations (i.e., exon 20 insertion mutations).

In some embodiments, the patient's cancer with CNS disease is identified as EGFR mutations that exclude ex20ins-mutations.

In some embodiments, the patient's cancer with CNS disease includes one or more mutation in the epidermal growth factor receptor (EGFR) gene e.g., as described herein.

In some embodiments, treatment according to the methods of this disclosure increases the overall survival (OS) time of a patient population as compared to a control patient population without the administration. In some embodiments, treatment according to the methods of this disclosure increases the progression-free survival (PFS) time of a patient population as compared to a control patient population without the administration.

In some embodiments, treatment according to the methods of this disclosure decreases the CNS metastasis of the patient's cancer. In some embodiments, treatment according to the methods of this disclosure reduces the progression of an existing CNS lesion, such as a brain metastasis. In some embodiments, treatment according to the methods of this disclosure reduces the size of an existing CNS lesion, such as a brain metastasis. In some embodiments, treatment according to the methods of this disclosure reduces the risk of new CNS lesion development.

The effectiveness of the treatment methods of this disclosure can be determined using any convenient patient assessment methods and outcome measures, e.g., as described herein in the experimental section, by comparing a pre-treatment or baseline assessment or measure with one taken post-treatment.

### 4.4. Definitions

Unless defined otherwise, all technical and scientific terms used herein have the same meaning as commonly understood by one of ordinary skill in the art to which this disclosure pertains. It is understood that the definitions provided herein are not intended to be mutually exclusive.

Ranges: throughout this disclosure, various aspects of the invention are presented in a range format. Ranges include the recited endpoints. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6, should be considered to have specifically disclosed subranges such as from 1 to 3, from 1to 4, from 1 to 5, from 2 to 4, from 2 to 6, from 3 to 6, etc. as well as individual number within that range, for example, 1, 2, 3, 4, 5, 5.3, and 6. This applies regardless of the breadth of the range.

In this disclosure, “comprises,” “comprising,” “containing,” “having,” “includes,” “including”, and linguistic variants thereof have the meaning ascribed to them in U.S. Patent law, permitting the presence of additional components beyond those explicitly recited.

Unless specifically stated or apparent from context, as used herein, the terms “a”, “an”, and “the” are understood to be singular or plural. That is, the articles “a” and “an” are used herein to refer to one or to more than one (i.e., to at least one) of the grammatical object of the article. By way of example, “an clement” means one clement or more than one clement.

Unless specifically stated or otherwise apparent from context, as used herein the term “about” is understood as within range of normal tolerance in the art, for example within 2 standard deviations of the mean, and is meant to encompass variations of ±20% or ±10%, more preferably ±5%, even more preferably ±1%, and still more preferably ±0.1% from the stated value. Where a percentage is provided with respect to an amount of a component or material in a composition, the percentage should be understood to be a percentage based on weight, unless otherwise stated or understood from the context.

The term “pre-treatment” means prior to the first administration of a DP1 receptor antagonist according the methods described herein. Pre-treatment does not exclude, and often includes, the prior administration of treatments other than a DPI receptor antagonist.

The term “post-treatment” means after the administration of a DPI receptor antagonist according the methods described herein. Post-treatment includes after any administration of a DP1 receptor antagonist at any dosage described herein. Post-treatment also includes after a bolus treatment phase of a DPI receptor antagonist, and also after a continuous administration of a DP1 receptor antagonist at any dosage described herein.

It should be understood that the order of steps or order for performing certain actions is immaterial so long as the present disclosure remain operable. Moreover, two or more steps or actions can be conducted simultaneously.

### 5. ADDITIONAL EMBODIMENTS

The disclosure is further described by the following non-limiting numbered clauses:
1. A method of treating cancer in a patient, wherein the patient's cancer has at least one mutation in the epidermal growth factor receptor (EGFR) gene, the method comprising:
orally administering to the patient a daily dose of 37.5-450 mg of NX-019 or a pharmaceutically acceptable salt thereof.
2. The method of clause 1 or 2, wherein the patient has lung cancer, head and neck cancer, or colon cancer.
3. The method of clause 1 or 2, wherein the patient has systemic non-small cell lung cancer (NSCLC).
4. The method of any one of clauses 1 to 3, wherein the patient exhibits systemic tumor regression after NX-019 administration.
5. The method of any one of clauses 1 to 4, wherein the patient's cancer is human epidermal growth factor receptor 2 positive (HER2⁺).
6. The method of of any one of clauses 1 to 5, wherein the at least one mutation comprises an insertion in the EGFR gene.
7. The method of clause 6, wherein the insertion is in Exon 20 of the EGFR gene.
8. The method of clause 6, wherein the insertion is not an insertion in Exon 20 of the EGFR gene.
9. The method of clause 6, wherein the insertion is in Exon 19 of the EGFR gene.
10. The method of any one of clauses 1 to 9, wherein the at least one mutation comprises a deletion in the EGFR gene.
11. The method of clause 10, wherein the deletion is in Exon 19 of the EGFR gene.
12. The method of of any one of clauses 1 to 11, wherein the at least one mutation comprises a point mutation in the EGFR gene.
13. The method of clause 12, wherein the at least one point mutation is located at one or more of amino acid positions L858, G719, L861, S768, and E709 of the EGFR gene.
14. The method of of any one of clauses 1 to 13, wherein the at least one mutation comprises a frameshift mutation in the EGFR gene.
15. The method of any one of clauses 1 to 14, wherein the patient's cancer is locally advanced.
16. The method of any one of clauses 1 to 14, wherein the patient's cancer is metastatic.
17. The method of clause 16, wherein the patient's cancer has bone metastasis.
18. The method of any one of clauses 1 to 17, wherein the patient has previously been treated with an EGFR-targeted therapy.
19. The method of clause 18, wherein the patient has previously been treated with osimertinib.
20. The method of any one of clauses 1 to 19, wherein the patient has not previously been treated with an EGFR-targeted therapy targeting an insertion in Exon 20.
21. The method of any one of clauses 1 to 20, wherein the daily oral dose of NX-019 or a pharmaceutically acceptable salt thereof, is at least 75 mg.
22. The method of clause 21, wherein the daily oral dose of NX-019 or a pharmaceutically acceptable salt thereof, is at least 150 mg.
23. The method of clause 22, wherein the daily oral dose of NX-019 or a pharmaceutically acceptable salt thereof, is at least 300 mg.
24. The method of clause 23, wherein the daily oral dose of NX-019 or a pharmaceutically acceptable salt thereof, is at least 375 mg.
25. The method of clause 21, wherein the daily oral dose of NX-019 or a pharmaceutically acceptable salt thereof, is 75-150 mg.
26. The method of clause 21, wherein the daily oral dose of NX-019 or a pharmaceutically acceptable salt thereof, is 150-300 mg.
27. The method of clause 21, wherein the daily oral dose of NX-019 or a pharmaceutically acceptable salt thereof, is 75 mg.
28. The method of clause 21, wherein the daily oral dose of NX-019 or a pharmaceutically acceptable salt thereof, is 150 mg.
29. The method of clause 21, wherein the daily oral dose of NX-019 or a pharmaceutically acceptable salt thereof, is 300 mg.
30. The method of clause 21, wherein the daily oral dose of NX-019 or a pharmaceutically acceptable salt thereof, is 375 mg.
31. The method of clause 21, wherein the daily oral dose of NX-019 or a pharmaceutically acceptable salt thereof, is 450 mg.
32. The method of any one of clauses 1 to 31, wherein the administration of the effective amount of NX-019 or a pharmaceutically acceptable salt thereof, increases the overall survival (OS) time of a patient population as compared to a control patient population without the administration.
33. The method of any one of clauses 1 to 32, wherein the administration of the effective amount of NX-019 or a pharmaceutically acceptable salt thereof, increases the progression-free survival (PFS) time of a patient population as compared to a control patient population without the administration.
34. A method of treating cancer in a patient having CNS metastasis, the method comprising:
orally administering to the patient a daily dose of 37.5-450 mg of NX-019 or a pharmaceutically acceptable salt thereof.
wherein the patient's cancer has central nervous system (CNS) metastasis and at least one mutation in the epidermal growth factor receptor (EGFR) gene.
35. The method of clause 34, wherein the patient's cancer does not have an insertion in Exon 20 of the EGFR gene.
36. The method of clause 34, wherein the patient's cancer has an insertion in Exon 20 of the EGFR gene.
37. The method of clause 36, wherein the patient has not previously been treated with an EGFR-targeted therapy targeting an insertion in Exon 20 of the EGFR gene.
38. The method of any one of clauses 34 to 37, wherein the patient's cancer is human epidermal growth factor receptor 2 positive (HER2⁺).
39. The method of any one of clauses 34 to 38, wherein the at least one mutation comprises an insertion in Exon 19 of the EGFR gene.
40. The method of any one of clauses 34 to 38, wherein the at least one mutation comprises a deletion in the EGFR gene.
41. The method of clause 40, wherein the deletion is in Exon 19 of the EGFR gene.
42. The method of of any one of clauses 34 to 41, wherein the at least one mutation comprises a point mutation in the EGFR gene.
43. The method of clause 42, wherein the at least one point mutation is located at one or more of amino acid positions L858, G719, L861, S768, and E709 of the EGFR gene.
44. The method of of any one of clauses 34 to 43, wherein the at least one mutation comprises a frameshift mutation in the EGFR gene.
45. The method of any one of clauses 34 to 37, wherein the patient's cancer has brain metastasis.
46. The method of any one of clauses 34 to 38, wherein the patient has lung cancer, head and neck cancer, or colon cancer.
47. The method of any one of clauses 34 to 39, wherein the patient has metastatic non-small cell lung cancer (NSCLC).
48. The method of any one of clauses 34 to 37, wherein the patient's cancer has asymptomatic leptomeningeal disease.
49. The method of any one of clauses 34 to 48, wherein the patient has previously been treated with an EGFR-targeted therapy.
50. The method of clause 49, wherein the patient has previously been treated with osimertinib.
51. The method of any one of clauses 34 to 50, wherein the patient has not previously been treated with an EGFR-targeted therapy targeting an insertion in Exon 20.
52. The method of any one of clauses 34 to 51, wherein the daily oral dose of NX-019 or a pharmaceutically acceptable salt thereof, is at least 75 mg.
53. The method of clause 52, wherein the daily oral dose of NX-019 or a pharmaceutically acceptable salt thereof, is at least 150 mg.
54. The method of clause 53, wherein the daily oral dose of NX-019 or a pharmaceutically acceptable salt thereof, is at least 300 mg.
55. The method of clause 54, wherein the daily oral dose of NX-019 or a pharmaceutically acceptable salt thereof, is at least 375 mg.
56. The method of clause 52, wherein the daily oral dose of NX-019 or a pharmaceutically acceptable salt thereof, is 75-150 mg.
57. The method of clause 52, wherein the daily oral dose of NX-019 or a pharmaceutically acceptable salt thereof, is 150-300 mg.
58. The method of clause 52, wherein the daily oral dose of NX-019 or a pharmaceutically acceptable salt thereof, is 75 mg.
59. The method of clause 52, wherein the daily oral dose of NX-019 or a pharmaceutically acceptable salt thereof, is 150 mg.
60. The method of clause 52, wherein the daily oral dose of NX-019 or a pharmaceutically acceptable salt thereof, is 300 mg.
61. The method of clause 52, wherein the daily oral dose of NX-019 or a pharmaceutically acceptable salt thereof, is 375 mg.
62. The method of clause 52, wherein the daily oral dose of NX-019 or a pharmaceutically acceptable salt thereof, is 450 mg.
63. The method of any one of clauses 34 to 62, wherein the administration of the effective amount of NX-019 or a pharmaceutically acceptable salt thereof, increases the overall survival (OS) time of a patient population as compared to a control patient population without the administration.
64. The method of any one of clauses 34 to 62, wherein the administration of the effective amount of NX-019 or a pharmaceutically acceptable salt thereof, increases the progression-free survival (PFS) time of a patient population as compared to a control patient population without the administration.
65. The method of any one of clauses 34 to 64, wherein the administration of the effective amount of NX-019 or a pharmaceutically acceptable salt thereof, decreases the CNS metastasis of the patient's cancer.
66. The method of any one of clauses 34 to 64, wherein the administration of the effective amount of NX-019 or a pharmaceutically acceptable salt thereof, reduces the progression of an existing CNS lesion.
67. The method of any one of clauses 34 to 64, wherein the administration of the effective amount of NX-019 or a pharmaceutically acceptable salt thereof, reduces the size of an existing CNS lesion.
68. The method of any one of clauses 34 to 64, wherein the administration of the effective amount of NX-019 or a pharmaceutically acceptable salt thereof, reduces the risk of new CNS lesion development.

### 6. EXAMPLES

The following examples are offered to illustrate the present disclosure and are not to be construed in any way as limiting the scope of the present technology. Any methods that are functionally equivalent are within the scope of the present technology. Various modifications of the present technology in addition to those described herein will become apparent to those skilled in the art from the foregoing description and accompanying figures. Such modifications fall within the scope of the appended claims. Unless otherwise stated, all temperatures are in degrees Celsius. Efforts have been made to ensure accuracy with respect to numbers used (e.g., amounts, temperatures, etc.), but some experimental errors and deviation should be allowed for. In the examples below, if an abbreviation is not defined, it has its generally accepted meaning.

### 6.1. Preclinical Studies of NX-019

Approved targeted EGFR therapies have limited efficacy toward many of the EGFR common driver mutations, such as L7858R, exon 19 mutations, and exon 20 insertion mutations. About 20%-50% of NSCLC patients develop brain metastases (BMs) during the course of the disease, but BMs are still poorly addressed by current tyrosine kinase inhibitors (TKIs). NX-019 is a potent, orally bioavailable, CNS penetrant, mutant selective EGFR inhibitor targeting a broad range of EGFR mutations including common activation mutations (e.g., exon 19 deletions and L858R), T790M, exon 20 insertions and other rare mutations.

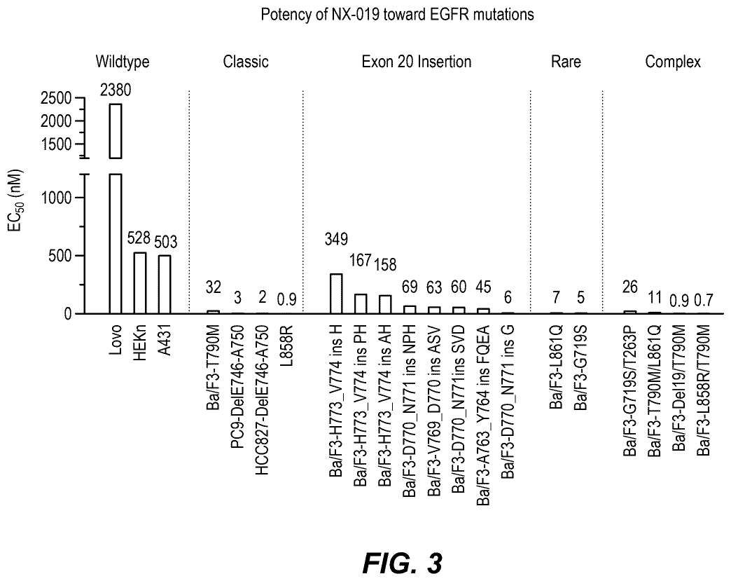

NX-019 is selective for mutant EGFR over wild-type EGFR. and broadly effective against EGFR for a wide range of mutations and mutational types. Mutant selectivity of >110 fold was shown in cancer cell line with mutant L858R (T1975) EGFR versus wild-type (Lovo) cancer cell line. This selectivity has also been shown in vitro across a large set of cell lines, which harbor a variety of EGFR mutations that lead to an activated or drug-resistant EGFR. Table 3 shows the in vitro cellular potency of NX-019 across wild-type and a wide variety of EGFR mutations. The results are also shown in FIG. 3, where Ba/F3 (engineered with specific EGFR mutations using standard protocols) or cancer cell lines, expressing wildtype or mutant EGFR, were treated with titrating concentrations of NX-019 and cell viability was measured after 3 days. NX-019 demonstrates enhanced potency in cell lines expressing all classes of EGFR mutations (e.g., classic, Exon 20 insertion, rare, complex) compared to cell lines with wildtype EGFR. These data demonstrate the broad potency and the mutant selectivity of NX-019.indicating the potential of NX-019 to treat patients with mutations not currently addressed by, or resistant to, current EGFR TKI therapies.

TABLE 3
Demonstration of in vitro Cell Potency by NX-019 With EGFR Mutations.

| | | | | EC50 |
|---|---|---|---|---|
| Mutation Category | Mutation(s) | Exon | Cell Line Model | (nm) |
| Wild Type | Wild Type | NA | Lovo | 2,300 |
| | Wild Type | NA | A431 | 503 |
| | Wild Type | NA | HEK | 528 |
| Common resistant | T790M | 20 | Ba/F3 | 32 |
| mutation | | | | |
| Classical | DelE746-A750 (Exon 19 | 19 | HCC827 | 2-3 |
| | del) | | | |
| | DelE746-A750 (Exon 19 | 19 | PC-9 | 3 |
| | del) | | | |
| | L858R | 21 | Ba/F3 | 0.9 |
| Exon 20 insertion | D770_N771 ins SVD | 20 | C2058 PDX line | 60 |
| | V769_D770 ins ASV | 20 | Ba/F3 | 63 |
| | 770_771 ins NPH | 20 | LU0387 PDX line | 69 |
| | D770_N771 ins G | 20 | CTG 2992 PDX | 5.5 |
| | | | line | |
| | A763_Y764 ins FQEA | 20 | Ba/F3 | 45 |
| | H773_V774 ins H | 20 | Ba/F3 | 349 |
| | H773_V774 ins PH | 20 | Ba/F3 | 167 |
| | H773_V774 ins AH | 20 | Ba/F3 | 158 |
| Rare mutations | G719S | 18 | Ba/F3 | 5 |
| | L861Q (L861X) | 21 | Ba/F3 | 7 |
| Complex | G719S/T263P | 18, | Ba/F3 | 26 |
| | | other | | |
| | Del19/T790M | 19 | Ba/F3 | 0.9 |
| | L858R/T790M | 20, 21 | Ba/F3 | 0.7 |
| | T790M/L861Q | 20, 21 | Ba/F3 | 11 |
| | T790M/L858R | 20, 21 | NCI-H1975 | 3-20 |

### 6.1.1. Assessment of NX-019 in Mouse Models of Cancer

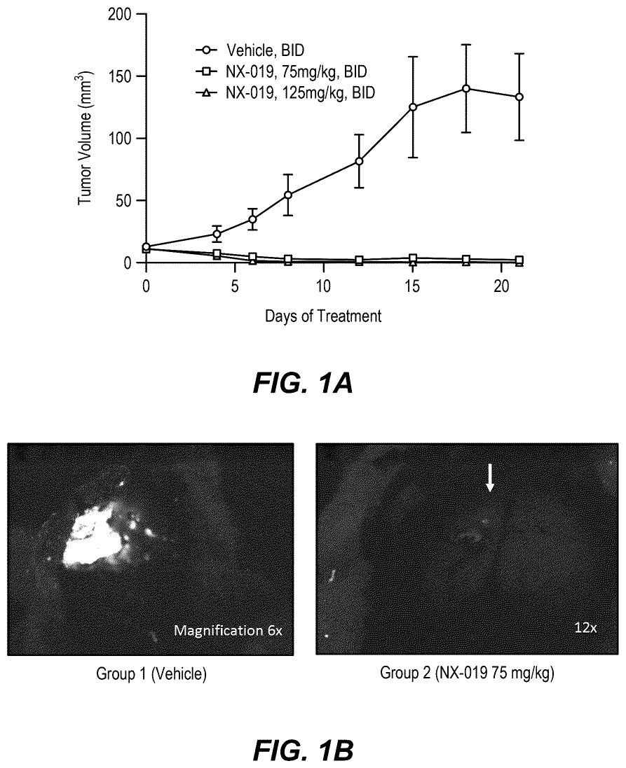

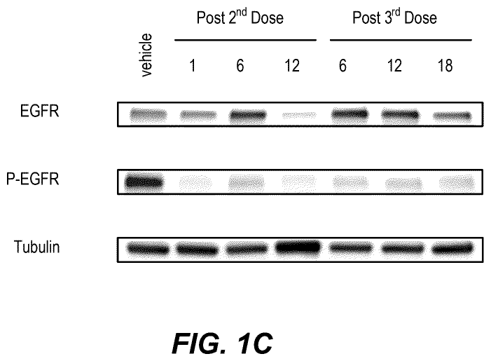

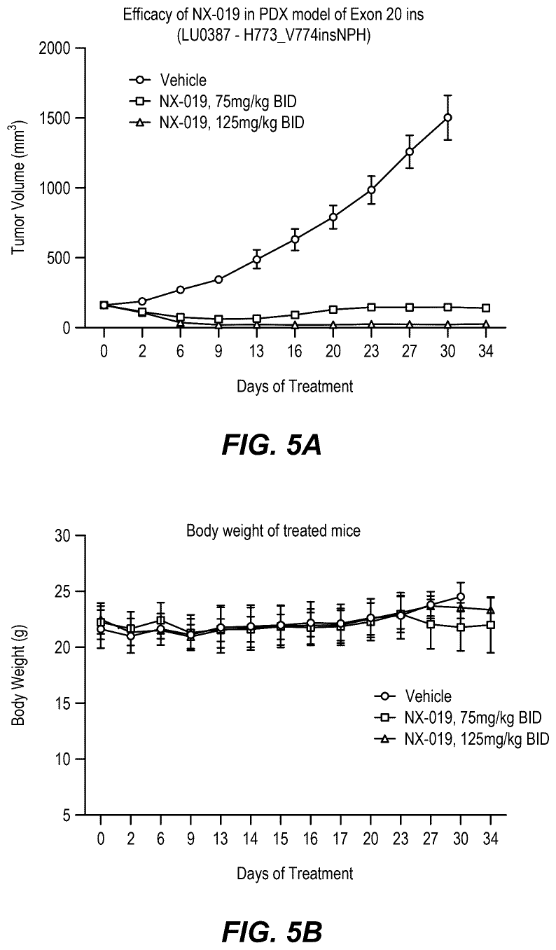

The potency of NX-019 has been demonstrated across a variety of mouse subcutaneous xenograft models, including lung orthotopic models, and a bone metastasis model. In all the xenograft models, NX-019 was well tolerated with no to minimal body weight loss during treatment (FIG. 5, panel B). In both in vitro and in vivo (FIG. 1, panels A-C; FIG. 5 panel A) models, NX-019 showed sustained down-regulation of phosphorylated EGFR in the treated cells and tumors. Referring to FIG. 5, PDX model Exon 20 ins (LU0387-H773_V774insNPH) cells were implanted subcutaneously in BALB/c nude mice. When tumors reached an average of 160mm³, NX-019 was dosed at 75 and 125 mg/kg in mice orally twice per day (PO BID). Tumor volume and body weight of the mice were measured and graphed (FIG. 5). NX-019 shows tumor regressions and cures at both tested doses (panel A) with no body weight effects (panel B). The efficacy of NX-019 toward CNS lesions in these xenograft brain models demonstrate tumor regressions and prolonged pharmacodynamic (PD) effects. NX-019 treatment resulted in tumor regressions and cures at both tested doses with no effects on body weight.

NX-019 is broadly active against EGFR mutations, shows selectivity for mutant EGFR, and has in vitro and in vivo activity in mutant driven EGFR models across mutational types and tumor locations.

### 6.1.2. Assessment of NX-019 on Mutant EGFR and Wildtype EGFR

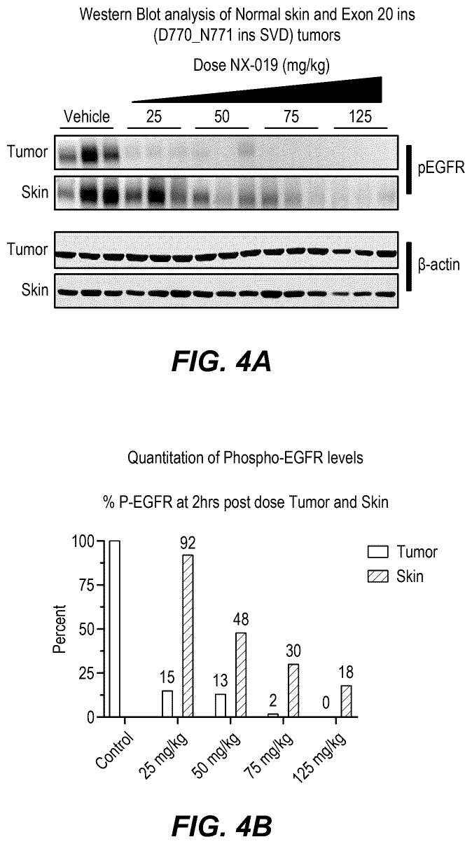

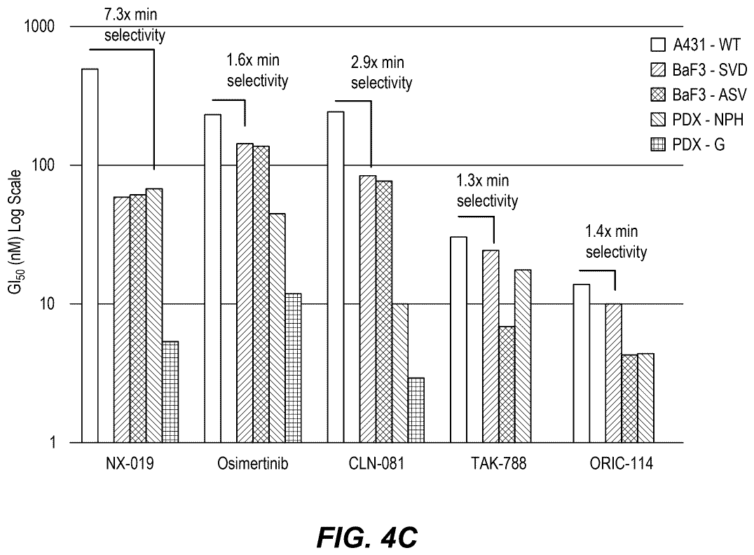

NX-019 shows selective inhibition of mutant EGFR over wildtype EGFR. Ba/F3 EGFR Exon 20 ins (D770_N771insSVD) expressing cells were subcutaneously implanted into BALB/c nude mice. When tumors reached an average of 310 mm³, mice were dosed with 25-125 mg/kg NX-019 PO BID×1. Tumor and skin samples were collected 2 hours post dosing and analyzed by western blot to detect levels of phosphorylated EGFR and β-actin. NX-019, at all tested dosages (25 mg/kg, 50 mg/kg, 75 mg/kg, 125 mg/kg) reduces tumor and skin phosphorylated EGFR expression at all tested dosages (FIG. 4, panel A). By contrast, β-actin protein level remains consistent withstand the dosage increase. Phosphorylated EGFR levels were quantitated and normalized to total EGFR and β-actin protein levels. As low as 25 mg/kg NX-019significantly reduces tumor EGFR level (FIG. 4, panel B). NX-019 demonstrated enhanced inhibitor of, and selectivity for, mutant EGFR in tumors compared to wildtype EGFR in skin samples.

Cell lines expressing wildtype EGFR or specific EGFR exon 20 insertion mutants were treated with titrating concentrations of NX-019 or other EGFR inhibitors and cell viability was measured (FIG. 4, panel C). NX-019 demonstrated improved EGFR exon 20 insertion mutation selectivity in comparison to other compounds tested.

These data demonstrate that NX-019 is highly selective for mutant EGFR over wild-type EGFR, and exhibits greater potency in cell lines with classic, exon 20 insertion, and other rare EGFR mutations compared to cells expressing wildtype EGFR. It is noted that classical EGFR dermatologic adverse events were not observed in preclinical animal models, which is in agreement with the improved selectivity profile of NX-019.

NX-019 is a broad-spectrum EGFR inhibitor with potent mutant EGFR inhibition activity, superior wild type EGFR selectivity, and excellent CNS penetration in animal models. NX-019 is a broad-spectrum EGFR inhibitor targeting both common and rare mutations including EGFR ex20ins, G719S, and L861Q. NX-019 exhibits selective activity towards mutant vs. wt. EGFR in preclinical studies.

### 6.1.3. Brain Penetrant Activity of NX-019

NX-019 has brain penetrant properties. Studies were conducted to demonstrate and characterize the ability of NX-019 to penetrate the CNS. In pharmacokinetic (PK) experiments to formally assess the distribution of NX 019 into the CNS compartment, a 100 mg/kg oral dose of NX-019 was administered to mice. The calculated free fraction level of NX-019 within the brain cortex exceeded 1 μm within 15 minutes and remained above 100 nm for approximately 4 hours. Progressive disease (PD) confirmations assessing impact on phospho-EGFR in an intracranial PC9 (exon 19del) model following 175 mg/kg NX-019 (per oral, QDx3) demonstrated robust down-regulation of phospho-EGFR for 12 to 18 hours post last dose (FIG. 1. panel C).

In dogs, cerebrospinal fluid (CSF) levels of NX-019 were assessed following a single oral dose of 40 mg/kg. Plasma Cₘₐₓ values were in the 2 μm to 3 μm range (total NX-019). Plasma free fraction at Cₘₐₓ was estimated to be ˜140 nm and was associated with a CSF Cₘₐₓ of ˜120 nm, suggesting an almost 1:1 relationship between plasma free fraction and CSF levels within 1 hour in dogs. Under these conditions, CSF levels greater than 40 nm were maintained for almost 4 hours.

A similar relationship between plasma free fraction and CSF levels was also demonstrated in rats. Taken together, these data indicate NX-019 enters the CNS and that CSF levels are closely correlated with plasma free fraction levels. CSF concentrations of NX-019 in human patient at Cₘₐₓ are anticipated to follow those of the plasma free fraction and track with the overall exposure/time profile of NX-019 in plasma indicating likely exposures able to inhibit EGFRm in brain metastases.

Substantial CNS exposure has been demonstrated in animal models, as well as potent anti-tumor activity toward intracranial xenografts with prolonged inhibition of EGFR signaling. Similar drug levels compared to plasma were measured in the brains of rats and mice. NX-109 was readily detectable in the CSF of cynomolgus monkeys, dogs and rats at concentrations similar to the estimated plasma free fraction.

### 6.1.4. Activity of NX-019 in Mouse Model of Brain Cancer

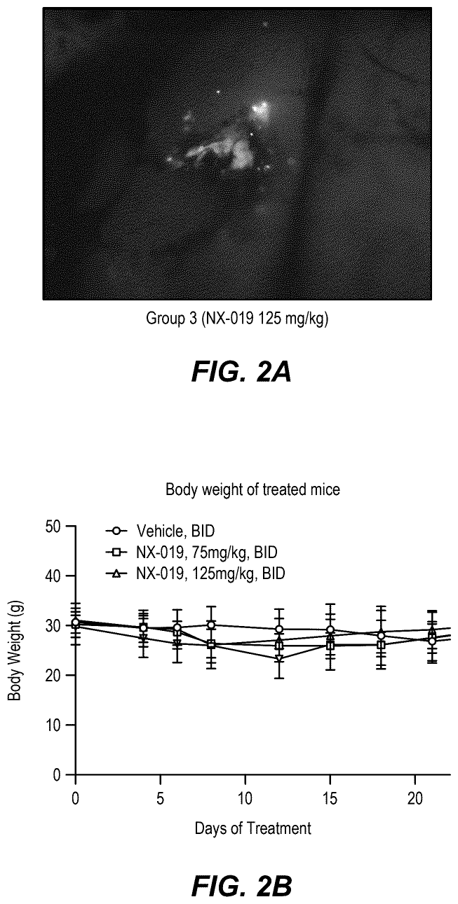

The potency of NX-019 has also been demonstrated in a NSCLC brain metastasis tumor model. FIG. 1 shows the efficacy of NX-019 in a mouse model of brain cancer (PC9-GFP) harboring an EGFR exon 19 insertional mutation (DelE746-A750) in the brain of NCr nu/nu mice. NX-019 was orally dosed at 75 and 125 or 150 mg/kg PO BID. Tumor volume was measured over time and graphed. The body weight of the mice was measured over time and graphed. NX-019 treatment caused complete regression of tumors as shown by the graph of tumor reduction (panel A), the photos of the GFP fluorescent tumors in control and treated mice at 75 mg/kg (panel B) at 22 days post-dose, and the down-regulation of phosphorylated EGFR in the tumors (panel C) (FIG. 1). FIG. 2 shows additional photo of GFP fluorescent tumor in treated mice at a higher dosage (125 mg/kg), which reduces tumor volume (GFP fluorescent) (panel A). NX-019 treatment resulted in tumor regressions and cures at both tested doses with no effects on body weight.

NX-019 was well tolerated with no to minimal body weight loss during treatment (FIG. 2, panel B). A companion study was completed looking at the PD effects of NX-019 in PC-9 CNS. NX-019 showed sustained down-regulation of phosphorylated EGFR in the tumors demonstrating on target pharmacodynamic inhibition of mutant EGFR (FIG. 1, panel C). Tumor lysates were analyzed by western blot using EGFR, phospho-EGFR and tubulin antibodies. NX-019showed sustained inhibition of EGFR phosphorylation at time points post dose (175 mg/kg) in western blot images of tumors probed with EGFR, P-EGFR and Tubulin antibodies (FIG. 1, panel C).

Thus, NX-019 is broadly active against EGFR mutations, shows selectivity for mutant EGFR, and has in vivo activity in mutant driven EGFR models of central nervous system (CNS) disease. NX-019 shows tumor regressions and cures at both tested doses with no body weight effects.

### 6.1.5. CNS/Brain Versus Plasma Exposure of NX-019 in Animal Models

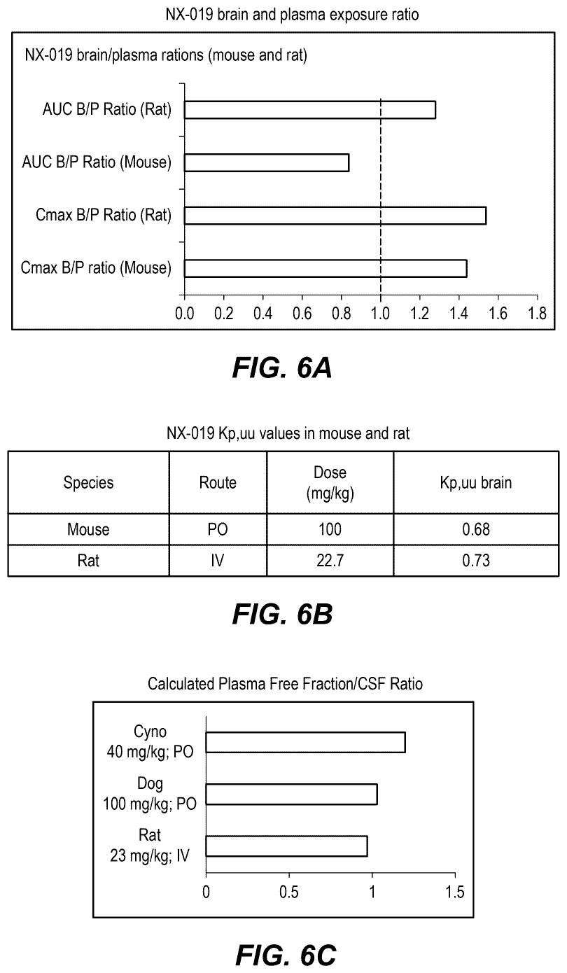

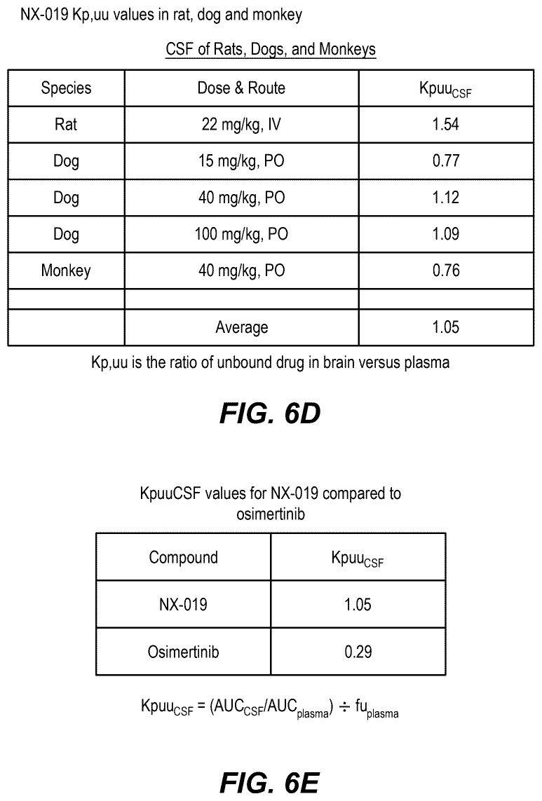

The significance of NX-019 brain and plasma exposure ratio was investigated. Pharmacokinetic exposure levels of NX-019 in plasma and brain were measured (FIG. 6, panel A). AUC of brain/plasma ratio in rat, Cₘₐₓ of brain/plasma in rat ration in rat, and Cₘₐₓ of brain/plasma in mouse have NX-019 a brain/plasma ratio of higher than 1 (vertical dotted line, FIG. 6, panel A). Kp,uu values of brain exposure levels of NX-019 were calculated for mouse and rat (FIG. 6, panel B). Kp,uu_{CSF} values of brain exposure levels of NX-019 were calculated for rat, dog, and cynomolgus monkey (FIG. 6 panel D). Kp,uucs value of NX-019 compared to Osimertinib (Colclough N, et al. Clin Cancer Res, 2021; 27:189-2021) is shown (FIG. 6, panel E). The results show a Kp,uu approaching 1, indicating excellent CNS exposure. The free fraction of NX-019 was measured in plasma and cerebrospinal fluid (CSF) in cynomolgus monkey, dog and rat (FIG. 6, panel C). The results show that NX-019 has similar levels of exposure in the CNS and plasma compartments which indicate that NX-019 is brain penetrant with excellent CNS exposure levels. Moreover, NX-019 was readily detectable in the CSF of rat, dog, and cynomolgus monkeys.

### 6.1.6. Conclusion

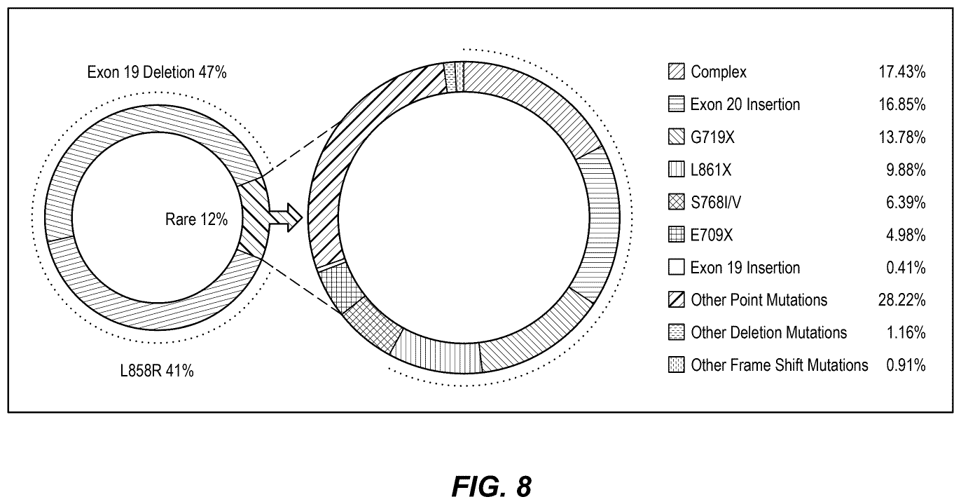

The data demonstrate that NX-019 is a potent and selective inhibitor of mutant EGFR, including classic, resistant and rare mutations such as exon 20 insertions (FIG. 8). Orally administered NX-019 is highly brain penetrant, active in orthotopic models of CNS metastasis at well tolerated doses, and shows prolonged inhibition of phospho-EGFR in implanted brain tumors. Additionally, NX-019 shows selective inhibitor of mutant EGFR whilst sparing wildtype EGFR function, both in vitro and in vivo. NX-019 displays high potency across a broad range of mutant EGFR models. NX-019 was optimized for enhanced CNS penetration and potency against a broad range of EGFR mutations. NX-019 is currently undergoing evaluation in a Phase 1 clinical trial (NCT05514496).

Preclinical in vitro and in vivo studies reveal that NX-019 has high potency across a broad range of mutant EGFR models indicating the potential of NX-019 to treat patients not currently covered or resistant to current EGFR TKI therapies. NX-019 is selective for mutant EGFR over wild-type EGFR and exhibits greater potency (7.3-200+ fold) in cell lines in cells with classic, exon20 insertion, and other rare mutations compared to cells expressing wild type EGFR. This improved mutant selectivity should facilitate improved tolerability at higher dose levels and greater systemic exposures. Studies in preclinical animal models (mouse, rat, dog) did not show classical EGFR dermatological findings, in agreement with the improved selectivity profile of NX-019. Studies to measure the CNS efficacy of NX-019 in xenograft brain models (such as PC9 brain orthotopic model) show tumor regressions and prolonged PD effects (e.g., inhibition of phospho-EGFR). CNS exposure studies in multiple preclinical species shows a Kp,uu of approximately 1 indicating excellent CNS exposure.

### 6.2. Preparation of NX-019 Formulation

N-(1-((R)-1-Acryloylazepan-3-yl)-7-chloro-6-(((S)-tetrahydrofuran-3-yl)oxy)-1H-benzo[d]imidazol-2-yl)-2-methylisonicotinamide (NX019) can be synthesized according to the synthetic methods described in Example 20 of U.S. Pat. No. 10,005,765. NX-019 free base can be isolated by crystallization in DCM and tert-butylmethyl ether (MTBE). Recrystallization of the NX019 free base product in ethanol/water gave crystalline Form I material. NX-019 free base Form I formulated in hydroxypropyl methylcellulose (HPMC) capsules is used in the study.

### 6.3. A Study of NX-019 in Patients with Advanced, Epidermal Growth Factor Receptor (EGFR) Mutant Cancer

### 6.3.1. Study Description and Objectives

Clinical Trial NCT05514496. This is a two-part, first-in-human, open-label study to determine the safety and tolerability of NX-019 and preliminary efficacy in patients with locally advanced or metastatic epidermal growth factor receptor (EGFR)-mutant cancer.

Dose Escalation (Part 1): The primary objective of Part 1 of this study is to evaluate the safety and tolerability of NX-019 and to determine the maximum tolerated dose (MTD)/Recommended Phase 2 Dose (RP2D).

Dose Escalation (Part 1): The secondary objective of Part 2 of this study is to:
1. To assess the pharmacokinetic (PK) profile of NX-019.
2. To determine anti-tumor activity of NX-019 as defined by Response Evaluation Criteria in Solid Tumors (RECIST) 1.1:

$$Objective response \text{} \text{} rate \text{} \text{} ( \text{} O R R ) \text{} ( O R R = complete response [ C R ] + partial response [ P R ] ) ; \qquad a .$$

b. Time to response (TTR);
c. Duration of response (DOR);
d. Disease control rate (DCR) (DCR=CR+PR+stable disease [SD]); and
e. Overall survival (OS) following initiation of NX-019 treatment.
3. To determine anti-tumor activity of NX-019 for central nervous system (CNS) disease per response assessment in neuro-oncology brain metastases (RANO-BM) guidance:
a. ORR CNS metastases;
b. TTR CNS metastases; and
c. DOR CNS metastases.

Dose Expansion (Part 2): The primary objective of Part 2 of this study is to confirm the safety and tolerability of NX-019 at the MTD/RP2D and, for each expansion cohort, the preliminary evidence of efficacy as measured by objective response rate (ORR).

Dose Expansion (Part 2): The secondary objective of Part 2 of this study is to:
1. To further assess the PK profile of NX 019.
2. To determine anti-tumor activity of NX 019 as defined by RECIST 1.1:
a. TTR;
b. DOR;

$$D C R ( D C R = C R + P R + S D ) ; \qquad c .$$

d. OS following initiation of NX-019 treatment.
3. To determine anti-tumor activity of NX-019 for CNS disease per RANO-BM guidance:
a. ORR CNS metastases;
b. TTR CNS metastases; and
c. DOR CNS metastases.

Exploratory-Dose Escalation (Part 1) and Dose Expansion (Part 2): the mutational profile of each tumor is characterized, and ORR is assessed by patient EGFR mutation type.

### 6.3.2. Study Design

This is a two-part, first-in-human, open-label, dose escalation and expansion study of orally administered NX-019 in patients with advanced, EGFR mutant cancer: Study Type: Interventional, Estimated Enrollment: 178 participants, Allocation: Non-Randomized, Intervention Model: Sequential Assignment, Primary Purpose: Treatment.

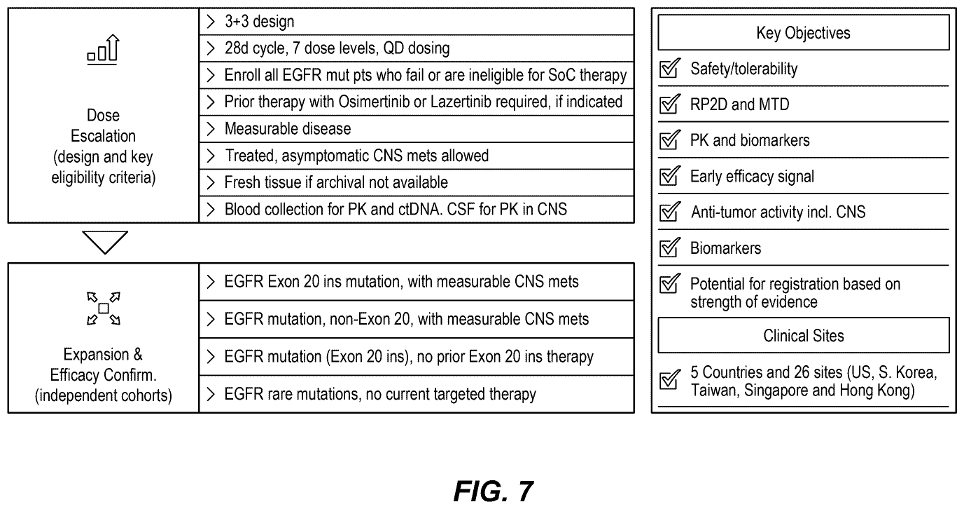

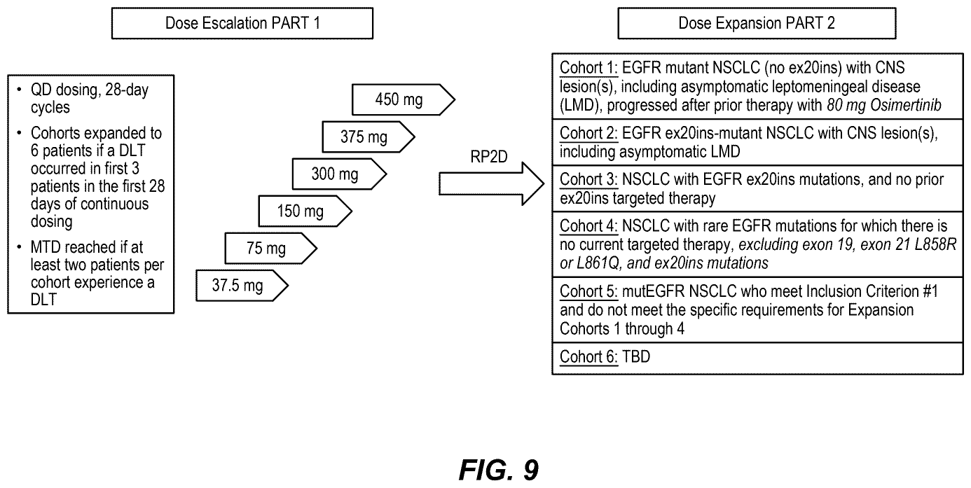

FIG. 7 and FIG. 9 show a flowchart of the study design. This study determines the safety and tolerability of NX-019 and preliminary efficacy in patients with locally advanced or metastatic epidermal growth factor receptor (EGFR)-mutant cancer. The study will include a Dose Escalation Part and a Dose Expansion Part. NX-019 is initially administered orally once daily (QD) in continuous 28-day cycles. Cohorts are expanded to 6 patients if a DLT occurred in first 3 patients in the first 28 days of continuous dosing. MTD is reached if at least two patients per cohort experience a DLT. Safety and tolerability of NX-019 are evaluated using the National Cancer Institute Common Terminology Criteria for Adverse Events (NCI CTCAE) version (v) 5.0. All patients are assessed for response using RECIST 1.1 and computed tomography (CT) or magnetic resonance (MR) imaging occurs 28 days within and every 8 weeks (+7 days) after Cycle (C) 1 Day (D) 1. Serial blood samples for determination of PK and pharmacodynamics, and circulating tumor DNA (ctDNA) are collected. An optional CSF sample is collected on C1D15 at 4 hours post-dose for assessment of NX-019 and any metabolite levels.

The Dose Escalation and Dose Expansion Parts each consist of a Screening Period, a Treatment Period (continuous 28-day cycles), an End of Treatment (EOT) Visit, a Safety Follow Up Visit, and a Long-Term Follow-Up (LTFU). Ongoing safety and disease assessments, disease status, survival, and subsequent anticancer therapy(ies) are assessed during the LTFU.

### 6.3.3. Arms and Intervention

Experimental: Part 1: NX-019 Dose Escalation. Patients are treated with NX-019 in multiple ascending cohorts. NX-019 is administered orally.

Experimental: Part 2: NX-019 Dose Escalation. Patients are treated with the MTD/RP2D of NX-019 in multiple ascending cohorts. NX-019 is administered orally.

### 6.3.4. Outcome Measures

Primary Outcome Measures include frequency, severity, and relatedness of treatment emergent adverse events (TEAEs), adverse events of special interest (AESIs), and serious adverse events (SAEs):
1. Part 1 and Part 2: Incidence of TEAEs [Time Frame: Up to 4.5 years].
2. Part 1 and Part 2: Incidence of AESIs [Time Frame: Up to 4.5 years].
3. Part 1 and Part 2: Incidence of SAEs [Time Frame: Up to 4.5 years].
4. Part 2: Objective response rate (ORR) [Time Frame: Up to 4.5 years].

Secondary Outcome Measures include plasma and cerebrospinal fluid (CSF) concentrations of NX-019 and calculated PK parameters, and parameters of anti-tumor activity/clinical benefit:
1. Part 1: Objective response rate of NX-019 [Time Frame: Up to 4.5 years].
2. Part 1 and Part 2: Plasma Concentration of NX-019 [Time Frame: Up to 43 days].
3. Part 1 and Part 2: Cerebrospinal Fluid (CSF) Concentration of NX-019 [Time Frame: Up to 43 days].
4. Part 1 and Part 2: Maximum Observed Serum Concentration (Cₘₐₓ) of NX-019 [Time Frame: Up to 43 days].
5. Part 1 and Part 2: Area under the concentration versus time curve (AUC) over a dosing interval (AUCtau) of NX-019 [Time Frame: Up to 43 days].
6. Part 1 and Part 2: AUC from time 0 to the time of last quantifiable plasma concentration (AUCO-t) of NX-019 [Time Frame: Up to 43 days].
7. Part 1 and Part 2: AUC from time 0 to infinity (AUCO-inf) of NX-019 [Time Frame: Up to 43 days].
8. Part 1 and Part 2: Percent of AUC extrapolated (AUC % extrap) of NX-019 [Time Frame: Up to 43 days].
9. Part 1 and Part 2: Terminal phase elimination half-life (t1/2) of NX-019 [Time Frame: Up to 43 days].
10. Part 1 and Part 2: Terminal phase elimination rate constant (Az) of NX-019 [Time Frame: Up to 43 days].
11. Part 1 and Part 2: Apparent plasma clearance (CL/F) of NX-019 [Time Frame: Up to 43 days].
12. Part 1 and Part 2: Apparent volume of distribution (Vd/F) of NX-019 [Time Frame: Up to 43 days].
13. Part 1 and Part 2: Accumulation index using Cmax (AICmax) and accumulation index using AUC (AIAUCO-inf) of NX-019 [Time Frame: Up to 43 days].
14. Part 1 and Part 2: Time to Response (TTR) [Time Frame: Up to 4.5 years].
15. Part 1 and Part 2: Duration of Response (DOR) [Time Frame: Up to 4.5 years].
16. Part 1 and Part 2: Disease Control Rate (DCR) [Time Frame: Up to 4.5 years].
17. Part 1 and Part 2: Overall Survival (OS) [Time Frame: Up to 4.5 years].
18. Part 1 and Part 2: Objective response rate for CNS (central nervous system) metastases [Time Frame: Up to 4.5 years].
19. Part 1 and Part 2: TTR for CNS (central nervous system) metastases [Time Frame: Up to 4.5 years].
20. Part 1 and Part 2: DOR for CNS (central nervous system) metastases [Time Frame: Up to 4.5 years].

### 6.3.5. Eligibility Criteria:

Ages Eligible for Study: 18 Years and older
Sexes Eligible for Study: All
Gender Based: No
Accepts Healthy Volunteers: No

### Inclusion Criteria:

1. Histologically confirmed, locally advanced, or metastatic EGFR-mutant cancer and has progressed on or are intolerant to all standard therapy.
2. Patients with non-small cell lung cancer (NSCLC) harboring a mutation that is sensitive to osimertinib must have received osimertinib prior to enrollment. Patients who have declined to receive platinum-based therapies may be enrolled. Patients with mutations for which there is no available inhibitor do not need to have received prior EGFR inhibitor therapy, but must have received alternate first line therapy.
3. Measurable disease per Response Evaluation Criteria in Solid Tumors (RECIST) 1.1 (evaluable disease acceptable for dose escalation part of study).
4. ≥18 years of age.
5. Life expectancy ≥3 months.
6. Adequate organ and bone marrow function defined by:
a. Absolute neutrophil count ≥1.5×10⁹/L (≥1500/mm³);
b. Platelet count ≥100×10⁹/L (≥100,000/mm³);
c. Total bilirubin ≤1.5×ULN; unless known Gilbert syndrome has been diagnosed;
d. AST and ALT ≤2.5×ULN or ≤5×ULN for patients with known liver metastases;
e. Measured (or calculated) creatinine clearance (glomerular filtration rate) ≥60 mL/min;
f. International Normalized Ratio or Prothrombin time (PT) or activated partial thromboplastin time (aPTT) ≤1.5×ULN. If a patient is receiving an anticoagulant therapy, PT, or aPTT must be within therapeutic range of intended use of anticoagulants.
7. All patients have a baseline magnetic resonance imaging (MRI) of the brain (or computed tomography [CT] with contrast if contraindicated), newly identified metastases are allowed if less than 1 cm, asymptomatic, with no neurologic changes or requirement for steroids and no need for immediate treatment in the opinion of the Investigator. Patients with treated, stable CNS metastases (including leptomeningeal carcinomatosis) are allowed if there is no evidence of progression for at least 2 weeks after CNS-directed treatment as ascertained by clinical examination and brain imaging, and the patient is receiving not more than 4 mg dexamethasone daily.
Note: This does not apply for patients in Expansion Cohorts 1 and 2 which specifically permit patients with progressing CNS lesions for assessment of patients with CNS disease; see Expansion Cohorts 1 and 2 below.
8. Resolution of any clinically significant toxic effects of prior therapy to Grade 0 or 1 according to the National Cancer Institute CTCAE v5.0 (exception of alopecia and Grade 2 peripheral neuropathy).
9. Eastern Cooperative Oncology Group (ECOG) performance status of ≤2.
10. Willingness of men and women of reproductive potential to observe conventional and effective birth control methods with failure rates of <1% for the duration of treatment and for 3 months following the last dose of study treatment.
11. A negative serum pregnancy test at Screening and a negative (serum or urine) pregnancy test within 72 hours before the first dose of study drug (female patients of childbearing potential only).
12. Willing and able to give informed consent and comply with protocol requirements for the duration of the study.

### Specific Inclusion Criteria for Expansion Cohorts:

To be eligible during the expansion part of the study, patients must meet the above inclusion criteria, and the criteria for 1 of the following cohorts:

### Expansion Cohort 1:

1. Patients with EGFR mutant NSCLC (excluding ex20ins mutations) with CNS lesion(s), including asymptomatic leptomeningeal disease, which have progressed or been detected after prior therapy.
2. Treatment regimen immediately preceding study enrollment must have included osimertinib 80 mg in combination or as a single agent;
3. Patients must have measurable CNS disease with ≥5 mm diameter by MRI with appropriate slice thickness (response will be assessed per RECIST using a 2 compartment model as defined in RANO-BM guidelines);
4. Patients that do not have disease requiring immediate treatment with surgery or radiation therapy;
5. Patients that are either not receiving steroids or are on a stable or decreasing dose of steroids (maximum allowed dose of 4 mg dexamethasone per day or equivalent).

### Expansion Cohort 2:

1. Patients with EGFR ex20ins-mutant NSCLC with CNS lesion(s), including asymptomatic leptomeningeal disease which have progressed or been detected after prior therapy.
2. Patients must have measurable CNS disease with >5 mm diameter by MRI with appropriate slice thickness (response will be assessed per RECIST using a 2 compartment model as defined in RANO-BM guidelines);
3. Patients that do not have disease requiring immediate treatment with surgery or radiation therapy;
4. Patients that are either not receiving steroids or are on a stable or decreasing dose of steroids (maximum allowed dose of 4 mg dexamethasone per day or equivalent).

### Expansion Cohort 3:

Patients with NSCLC and EGFR ex20ins mutations, and no prior ex20ins mutation targeted therapy.

### Expansion Cohort 4:

Patients with NSCLC and rare EGFR mutations for which there is no current targeted therapy, excluding exon 19, exon 21 L858R or L861Q, and ex20ins mutations.

### Expansion Cohort 5:

Patients with EGFR mutant NSCLC who meet Inclusion Criterion #1 and do not meet the specific requirements for Expansion Cohorts 1 through 4, after discussion with sponsor.

### Exclusion Criteria:

Patients who meet any of the following criteria will be excluded from participation in the study:
1. Known C797X EGFR mutations or known second driver of disease.
2. Received systemic anticancer chemotherapy, targeted agents, antibody therapy for cancer, immunotherapy for cancer, hormonal therapy or an investigational agent within 2 weeks or 5 half-lives (whichever is shorter) prior to start of study drug treatment.
3. Major surgery within 3 weeks prior to start of study drug treatment.
4. Radiation therapy within 4 weeks prior to start of study drug treatment.
5. Severe or unstable cardiac conditions within 6 months prior to starting study drug treatment.
6. Severe or unstable medical condition including uncontrolled diabetes or unstable psychiatric condition.
7. Dependent on contact lenses (unable to wear eyeglasses) or unable to comply with ophthalmic guidance.
8. History of interstitial lung disease, radiation pneumonitis which required systemic steroid therapy, or other significant lung disease.
9. Has a history of another active malignancy (a second cancer).
10. Active infection requiring systemic therapy.
11. Known human immunodeficiency virus (HIV), hepatitis B virus (HBV) (i.e., hepatitis B surface antigen-positive), or hepatitis C virus (HCV) (i.e., detectable HCV ribonucleic acid [RNA]).
12. Active gastrointestinal disease (e.g., Crohn's disease, ulcerative colitis, or short gut syndrome) or conditions that may impact drug absorption.
13. Pregnant or breastfeeding.
14. Is using a strong CYP3A inhibitor or inducer.
15. Any other condition, including significant skin or nail disease, that in the opinion of the Investigator would place the patient at an unacceptable risk or cause the patient to be unlikely to fully participate or comply with study procedures.

### 6.3.6. Procedures

Study procedures follow a schedule of assessments of the patients during the study. Procedure for selected tests are shown below.

### 6.3.6.1 Tumor Measurements

Tumors are assessed based on RECIST 1.1. CNS disease is assessed by RECIST using a two compartment model (CNS and non-CNS) according to RANO-BM guidelines. Baseline disease assessment with radiographic tumor measurements using CT or MR imaging of chest, abdomen, and pelvis or any other areas with suspected disease involvement within 28 days of C1D1. All patients must have brain imaging (MR imaging preferred, CT with contrast is acceptable if MRI contraindicated) during Screening. For each modality, intravenous (IV) and oral contrast should be utilized (chest CT does not require IV contrast) unless there is a clear contraindication (e.g., decreased renal function or allergy that cannot be addressed with standard prophylactic treatments). On-study scans should be performed every 8 weeks (+7 days) after C1D1 for the first year and every 12 weeks (+7 days) thereafter, including imaging of the chest, abdomen, pelvis, and brain, or any other areas of known disease at baseline, using the same modality(ies) as used for baseline imaging assessment until PD, withdrawal of consent, or initiation of a new anticancer therapy(ies).

### 6.3.7. Study Treatments

NX-019 is provided in 37.5 and 75 mg capsules that are provided in bottles of 35capsules/bottle. The pharmacist dispenses capsules or bottles to the patient in an amount necessary to allow for outpatient administration at the assigned dose level. Dosing is intended to be fixed (i.e., not weight-based or BSA-based).

Capsules are stored at a controlled room temperature (between 15° C. and 30° C. [59° F. to 86° F.). Currently available data support a retest period of 12 months for the packaged drug product, which can be extended with additional stability data.

### 6.3.7.1 NX-019 Administration: General Dosing Instructions

NX-019 is initially administered orally QD in continuous 28-day cycles. Patients fast for 2 hours before and 1 hour after NX-019 administration. NX-019 is administered with approximately 8 ounces (approximately 236 mL) of water. Dosing for an individual is at a consistent time each day. NX-019 is swallowed whole and taken in a consistent fashion.

### Cycle 1:

Patients begin dosing on C1D1 according to their assigned cohort. Serial blood samples for PK monitoring are collected and the patient is monitored for safety. An optional CSF sample is collected on C1D15 at 4 hours post-dose for assessment of NX-019 and any metabolite levels. Cycle length is 28 days during both the Dose Escalation and Dose Expansion parts; the DLT period during Part 1 will consist of Days 1-28 of C1.

### Cycles 2 and Higher:

In accordance with the assigned dose level (or for Part 2, the RP2D), each patient receives NX-019 QD in continuous 28-day cycles until progressive disease (PD), unacceptable toxicity, or other reasons for treatment discontinuation. Patients with documented PD may be allowed to continue NX-019 if the patient is tolerating treatment and, the patient is deriving clinical benefit from continuing study treatment and continuation of treatment.

### Dose Delays/Modifications

Cycles are 28 days in duration regardless of dose interruption unless the dose interruption includes D1 of the next cycle in which case the next cycle will start with resumption of study drug. In this situation, disease assessments continue to follow the original schedule.

Toxicities such as nausea, vomiting, diarrhea, dizziness, or dehydration related to these symptoms, may be managed with increased supportive care, including hydration, electrolyte repletion, and the use of anti-emetics such as 5-hydroxytriptamine antagonists and/or anti diarrheals such as loperamide as appropriate.

A patient who experiences a clinically significant adverse event (i.e., >Grade 2 or more than 1 grade change from baseline if baseline is Grade 2 or above) may have NX-019 dosing interrupted for up to 28 days to evaluate the adverse event and to allow for recovery (to Grade 1or baseline level). Upon recovery, the patient may restart therapy if it is considered in his/her best interest to continue therapy. Upon restarting, the patient may have the dose reduced by at least 1 dose level (Table). If the adverse event does not recover to Grade 1 or less within 28days (or baseline), the patient will have treatment permanently discontinued, unless there is a compelling clinical rationale for additional dose reduction(s) articulated by the Investigator and approved by the Sponsor. For each patient, a maximum of 2 dose reductions are allowed, unless there is a compelling clinical rationale for additional dose reduction(s).

TABLE 2
Suggested Toxicity Management

| Dose Level | Dose of NX-019 |
|---|---|
| Starting dose | Starting dose level |
| First dose reduction | Reduce dose by 1 dose level |
| Second dose reduction | Reduce dose by 1 additional dose level |
| Third dose reduction and | Reduce dose by 1 additional dose level |
| beyond^{[1]} | |

Only permitted if there is a compelling clinical rationale articulated by the Investigator and approved by the Sponsor.

### 6.3.8. Safety Assessments

An adverse event is defined as any untoward medical occurrence in a clinical investigation patient administered a pharmaceutical product, which does not necessarily have a causal relationship with this treatment. An adverse event can therefore be any unfavorable and/or unintended sign (including an abnormal laboratory finding), symptom, or disease temporally associated with the use of an investigational medicinal product, whether or not related to the investigational medicinal product. All adverse events, including observed or volunteered problems, complaints, or symptoms, are recorded.

The safety profile is based on adverse events (including DLTs, AESIs, and SAEs), physical examination findings (including ECOG performance status), ophthalmic examinations, vital sign measurements, clinical laboratory measurements, and ECG recordings. Safety analyses in general are descriptive and presented in a tabular format with the appropriate summary statistics. Adverse events will be coded using the Medical Dictionary for Regulatory Activities (MedDRA).

### 7. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All publications, patents, patent applications, and other documents cited in this application, including U.S. Provisional Appl. No. 63/398,192, U.S. Provisional Appl. No. 63/385,388, U.S. Provisional Appl. No. 63/484,791, and U.S. Provisional Appl. No. 63/504,416, are hereby incorporated by reference in their entireties for all purposes to the same extent as if each individual publication, patent, patent application, or other document were individually indicated to be incorporated by reference for all purposes.

What is claimed is:

1. A method of treating cancer in a patient, wherein the patient's cancer has at least one mutation in the epidermal growth factor receptor (EGFR) gene, the method comprising:
orally administering to the patient a daily dose of 37.5-450 mg of NX-019 or a pharmaceutically acceptable salt thereof.

2. The method of claim 1 or 2, wherein the patient has lung cancer, head and neck cancer, or colon cancer.

3. The method of claim 1 or 2, wherein the patient has systemic non-small cell lung cancer (NSCLC).

4. The method of any one of claims 1 to 3, wherein the patient exhibits systemic tumor regression after NX-019 administration.

5. The method of any one of claims 1 to 4, wherein the patient's cancer is human epidermal growth factor receptor 2 positive (HER2⁺).

6. The method of of any one of claims 1 to 5, wherein the at least one mutation comprises an insertion in the EGFR gene.

7. The method of claim 6, wherein the insertion is in Exon 20 of the EGFR gene.

8. The method of claim 6, wherein the insertion is not an insertion in Exon 20 of the EGFR gene.

9. The method of claim 6, wherein the insertion is in Exon 19 of the EGFR gene.

10. The method of any one of claims 1 to 9, wherein the at least one mutation comprises a deletion in the EGFR gene.

11. The method of claim 10, wherein the deletion is in Exon 19 of the EGFR gene.

12. The method of of any one of claims 1 to 11, wherein the at least one mutation comprises a point mutation in the EGFR gene.

13. The method of claim 12, wherein the at least one point mutation is located at one or more of amino acid positions L858, G719, L861, S768, and E709 of the EGFR gene.

14. The method of of any one of claims 1 to 13, wherein the at least one mutation comprises a frameshift mutation in the EGFR gene.

15. The method of any one of claims 1 to 14, wherein the patient's cancer is locally advanced.

16. The method of any one of claims 1 to 14, wherein the patient's cancer is metastatic.

17. The method of claim 16, wherein the patient's cancer has bone metastasis.

18. The method of any one of claims 1 to 17, wherein the patient has previously been treated with an EGFR-targeted therapy.

19. The method of claim 18, wherein the patient has previously been treated with osimertinib.

20. The method of any one of claims 1 to 19, wherein the patient has not previously been treated with an EGFR-targeted therapy targeting an insertion in Exon 20.

21. The method of any one of claims 1 to 20, wherein the daily oral dose of NX-019 or a pharmaceutically acceptable salt thereof, is at least 75 mg.

22. The method of claim 21, wherein the daily oral dose of NX-019 or a pharmaceutically acceptable salt thereof, is at least 150 mg.

23. The method of claim 22, wherein the daily oral dose of NX-019 or a pharmaceutically acceptable salt thereof, is at least 300 mg.

24. The method of claim 23, wherein the daily oral dose of NX-019 or a pharmaceutically acceptable salt thereof, is at least 375 mg.

25. The method of claim 21, wherein the daily oral dose of NX-019 or a pharmaceutically acceptable salt thereof, is 75-150 mg.

26. The method of claim 21, wherein the daily oral dose of NX-019 or a pharmaceutically acceptable salt thereof, is 150-300 mg.

27. The method of claim 21, wherein the daily oral dose of NX-019 or a pharmaceutically acceptable salt thereof, is 75 mg.

28. The method of claim 21, wherein the daily oral dose of NX-019 or a pharmaceutically acceptable salt thereof, is 150 mg.

29. The method of claim 21, wherein the daily oral dose of NX-019 or a pharmaceutically acceptable salt thereof, is 300 mg.

30. The method of claim 21, wherein the daily oral dose of NX-019 or a pharmaceutically acceptable salt thereof, is 375 mg.

31. The method of claim 21, wherein the daily oral dose of NX-019 or a pharmaceutically acceptable salt thereof, is 450 mg.

32. The method of any one of claims 1 to 31, wherein the administration of the effective amount of NX-019 or a pharmaceutically acceptable salt thereof, increases the overall survival (OS) time of a patient population as compared to a control patient population without the administration.

33. The method of any one of claims 1 to 32, wherein the administration of the effective amount of NX-019 or a pharmaceutically acceptable salt thereof, increases the progression-free survival (PFS) time of a patient population as compared to a control patient population without the administration.

34. A method of treating cancer in a patient having CNS metastasis, the method comprising:
orally administering to the patient a daily dose of 37.5-450 mg of NX-019 or a pharmaceutically acceptable salt thereof,
wherein the patient's cancer has central nervous system (CNS) metastasis and at least one mutation in the epidermal growth factor receptor (EGFR) gene.

35. The method of claim 34, wherein the patient's cancer does not have an insertion in Exon 20 of the EGFR gene.

36. The method of claim 34, wherein the patient's cancer has an insertion in Exon 20 of the EGFR gene.

37. The method of claim 36, wherein the patient has not previously been treated with an EGFR-targeted therapy targeting an insertion in Exon 20 of the EGFR gene.

38. The method of any one of claims 34 to 37, wherein the patient's cancer is human epidermal growth factor receptor 2 positive (HER2⁺).

39. The method of any one of claims 34 to 38, wherein the at least one mutation comprises an insertion in Exon 19 of the EGFR gene.

40. The method of any one of claims 34 to 38, wherein the at least one mutation comprises a deletion in the EGFR gene.

41. The method of claim 40, wherein the deletion is in Exon 19 of the EGFR gene.

42. The method of of any one of claims 34 to 41, wherein the at least one mutation comprises a point mutation in the EGFR gene.

43. The method of claim 42, wherein the at least one point mutation is located at one or more of amino acid positions L858, G719, L861, S768, and E709 of the EGFR gene.

44. The method of of any one of claims 34 to 43, wherein the at least one mutation comprises a frameshift mutation in the EGFR gene.

45. The method of any one of claims 34 to 37, wherein the patient's cancer has brain metastasis.

46. The method of any one of claims 34 to 38, wherein the patient has lung cancer, head and neck cancer, or colon cancer.

47. The method of any one of claims 34 to 39, wherein the patient has metastatic non-small cell lung cancer (NSCLC).

48. The method of any one of claims 34 to 37, wherein the patient's cancer has asymptomatic leptomeningeal disease.

49. The method of any one of claims 34 to 48, wherein the patient has previously been treated with an EGFR-targeted therapy.

50. The method of claim 49, wherein the patient has previously been treated with osimertinib.

51. The method of any one of claims 34 to 50, wherein the patient has not previously been treated with an EGFR-targeted therapy targeting an insertion in Exon 20.

52. The method of any one of claims 34 to 51, wherein the daily oral dose of NX-019 or a pharmaceutically acceptable salt thereof, is at least 75 mg.

53. The method of claim 52, wherein the daily oral dose of NX-019 or a pharmaceutically acceptable salt thereof, is at least 150 mg.

54. The method of claim 53, wherein the daily oral dose of NX-019 or a pharmaceutically acceptable salt thereof, is at least 300 mg.

55. The method of claim 54, wherein the daily oral dose of NX-019 or a pharmaceutically acceptable salt thereof, is at least 375 mg.

56. The method of claim 52, wherein the daily oral dose of NX-019 or a pharmaceutically acceptable salt thereof, is 75-150 mg.

57. The method of claim 52, wherein the daily oral dose of NX-019 or a pharmaceutically acceptable salt thereof, is 150-300 mg.

58. The method of claim 52, wherein the daily oral dose of NX-019 or a pharmaceutically acceptable salt thereof, is 75 mg.

59. The method of claim 52, wherein the daily oral dose of NX-019 or a pharmaceutically acceptable salt thereof, is 150 mg.

60. The method of claim 52, wherein the daily oral dose of NX-019 or a pharmaceutically acceptable salt thereof, is 300 mg.

61. The method of claim 52, wherein the daily oral dose of NX-019 or a pharmaceutically acceptable salt thereof, is 375 mg.

62. The method of claim 52, wherein the daily oral dose of NX-019 or a pharmaceutically acceptable salt thereof, is 450 mg.

63. The method of any one of claims 34 to 62, wherein the administration of the effective amount of NX-019 or a pharmaceutically acceptable salt thereof, increases the overall survival (OS) time of a patient population as compared to a control patient population without the administration.

64. The method of any one of claims 34 to 62, wherein the administration of the effective amount of NX-019 or a pharmaceutically acceptable salt thereof, increases the progression-free survival (PFS) time of a patient population as compared to a control patient population without the administration.

65. The method of any one of claims 34 to 64, wherein the administration of the effective amount of NX-019 or a pharmaceutically acceptable salt thereof, decreases the CNS metastasis of the patient's cancer.

66. The method of any one of claims 34 to 64, wherein the administration of the effective amount of NX-019 or a pharmaceutically acceptable salt thereof, reduces the progression of an existing CNS lesion.

67. The method of any one of claims 34 to 64, wherein the administration of the effective amount of NX-019 or a pharmaceutically acceptable salt thereof, reduces the size of an existing CNS lesion.

68. The method of any one of claims 34 to 64, wherein the administration of the effective amount of NX-019 or a pharmaceutically acceptable salt thereof, reduces the risk of new CNS lesion development.
